(19) United States
(12) Patent Application Publication
(10) Pub. No.: US 2025/0333504 A1
(43) Pub. Date: Oct. 30, 2025

(54) TREATMENT

(71) Applicant: IMMUNOCORE LIMITED, Abingdon, Oxfordshire (GB)

(72) Inventors: Mohammed DAR, Abingdon, Oxfordshire (GB); Shannon MARSHALL, Abingdon, Oxfordshire (GB); Shaad Essa ABEDIN, Abingdon, Oxfordshire (GB)

(21) Appl. No.: 18/716,126

(22) PCT Filed: Nov. 30, 2022
(86) PCT No.: PCT/EP2022/083952

§ 371 Date: Jun. 3, 2024

Related U.S. Application Data
(60) Provisional application No. 63/285,044, filed on Dec. 1, 2021.
(60) Provisional application No. 63/374,222, filed on Aug. 31, 2022.

(51) Int. Cl. C07K 16/28; A61K 9/00; A61K 38/00; A61P 35/00; C07K 14/725

(52) U.S. Cl. CPC C07K 16/2809; A61K 9/0019; A61P 35/00; C07K 14/7051; C07K 16/2818; C07K 16/2827; A61K 38/00; C07K 2317/565; C07K 2319/00

(57) ABSTRACT

Methods are presented for the administration of a TCR-anti-CD3 fusion molecule to treat patients who have a PRAME positive cancer. The methods comprise administering an TCR-anti-CD3 fusion molecule to a patient intravenously and comprise administration of (a) at least one first dose in the range of from 5-40 μg; (b) at least one second dose in the range of from 15-80 μg; and then (c) at least one third dose in the range of from 60-400 μg, wherein the second dose is higher than the first dose and the third dose is higher than the second dose, and wherein doses are administered every 6-8 days.

The present invention relates to the treatment of cancer, particularly PRAME positive cancers. In particular, it relates to a dosage regimen for a T cell redirecting bispecific therapeutic (TCR-anti-CD3 fusion molecule) comprising a T cell receptor (TCR) that binds the HLA-A*02 restricted peptide SLLQHLIGL (SEQ ID NO: 1), fused to an anti-CD3 scFv.

PRAME, or Preferentially Expressed Antigen In Melanoma, was first identified as an antigen that is over expressed in melanoma (Ikeda et al Immunity. 1997 February; 6 (2): 199-208); it is also known as CT130, MAPE, OIP-4 and has Uniprot accession number P78395. The protein functions as a repressor of retinoic acid receptor signalling (Epping et al., Cell. 2005 Sep. 23; 122 (6): 835-47). PRAME belongs to the family of germline-encoded antigens known as cancer testis antigens. Cancer testis antigens are attractive targets for immunotherapeutic intervention since they typically have limited or no expression in normal adult tissues. PRAME is expressed in a number of solid tumours as well as in leukaemias and lymphomas (Doolan et al Breast Cancer Res Treat. 2008 May; 109 (2): 359-65; Epping et al Cancer Res. 2006 Nov. 15; 66 (22): 10639-42; Ercolak et al Breast Cancer Res Treat. 2008 May; 109 (2): 359-65; Matsushita et al Leuk Lymphoma. 2003 March; 44 (3): 439-44; Mitsuhashi et al Int. J Hematol. 2014; 100 (1): 88-95; Proto-Sequeire et al Leuk Res. 2006 November; 30 (11): 1333-9; Szczepanski et al Oral Oncol. 2013 February; 49 (2): 144-51; Van Baren et al Br J Haematol. 1998 September; 102 (5): 1376-9). PRAME targeting therapies of the invention may be particularly suitable for treatment of cancers including, but not limited to, melanoma, lung cancer, breast cancer, ovarian cancer, endometrial cancer, oesophageal cancer, bladder cancer, head and neck cancer, uterine cancer, Acute myeloid leukemia, chronic myeloid leukemia, and Hodgkin's lymphoma.

The peptide SLLQHLIGL (SEQ ID NO: 1) corresponds to amino acids 425-433 of the full length PRAME protein and is presented on the cell surface in complex with HLA-A*02 (Kessler et al., J Exp Med. 2001 Jan. 1; 193 (1): 73-88). This peptide-HLA complex provides a useful target for TCR-based immunotherapeutic intervention.

WO/2018/234319 describes TCRs that bind to the SLLQHLIGL-HLA-A*02 complex. The TCRs are mutated relative to a native PRAME TCR alpha and/or beta variable domains to have improved binding affinities for, and/or binding half-lives, for the complex, and can be associated (covalently or otherwise) with a therapeutic agent. One such therapeutic agent is an anti-CD3 antibody, or a functional fragment or variant of said anti-CD3 antibody such as a single chain variable fragment (scFv). The anti-CD3 antibody or fragment may be covalently linked to the C- or N-terminus of the alpha or beta chain of the TCR. The resulting molecule is a TCR bispecific.

TCR bispecific proteins redirect polyclonal T cells to target peptides derived from intra- or extra-cellular disease associated antigens and presented on the cell surface in complex with an HLA molecule. This approach has been tested clinically in the context of a different antigen with a TCR bispecific protein targeting a HLA-A*02 restricted peptide from gp100 and CD3 (tebentafusp). Administration of this molecule provided an OS benefit in uveal melanoma (Nathan P, et al. Overall Survival Benefit with Tebentafusp in Metastatic Uveal Melanoma. N Engl J Med 2021; 385:1196-1206). However, no such TCR bispecific proteins targeting PRAME have been tested clinically.

IMC-F106C is a T cell redirecting bispecific therapeutic agent comprising a soluble affinity enhanced TCR that binds to the SLLQHLIGL peptide-HLA-A*02 complex, fused to an anti-CD3 scFv. The targeting end of IMC-F106C (the soluble TCR) binds to a peptide fragment of the PRAME antigen presented by HLA-A*02 on the surface of cancer cells. HLA molecules are polymorphic; approximately 47% of Caucasian individuals in the US and European countries express the HLA-A*02 genotype with the HLA-A*02:01 allele detected in more than 95% of HLA-A*02-positive individuals. The effector end of IMC-F106C (anti-CD3 scFv) can bind to CD3 on any T cell, redirecting the T cell to produce effector cytokines and/or kill the cell presenting the target. In addition, IMC-F106C-mediated tumour lysis may prime an endogenous anti-tumour immune response. ImmTAC® (Immune Mobilizing Monoclonal TCRs Against Cancer) molecules such as IMC-F106C are highly potent molecules, with redirection of T-cell activity observed against tumour cell lines presenting as few as 10 to 50 target peptide: HLA complexes. IMC-F106C has been shown to selectively redirect T cell activity in the presence of HLA-A*02:01-positive/PRAME-positive cell lines, leading to T cell activation and killing of PRAME-positive cancer cells, at concentrations as low as 1 pM to 10 pM. As described above, the HLA-A*02 restricted peptide SLLQHLIGL (SEQ ID NO: 1) is derived from the germline cancer antigen PRAME. IMC-F106C has a TCR alpha chain amino acid sequence of SEQ ID NO: 14 and a TCR beta chain-anti-CD3 amino acid sequence of SEQ ID NO: 16.

In a first aspect, the present invention provides a TCR-anti-CD3 fusion molecule comprising:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating PRAME positive cancer in a patient comprising administering the TCR-anti-CD3 fusion molecule to said patient intravenously, wherein the method comprises administration of:
(a) at least one first dose in the range of from 5-40 μg;
(b) at least one second dose in the range of from 15-80 μg; and then
(c) at least one third dose in the range of from 60-400 μg,
wherein the second dose is higher than the first dose and the third dose is higher than the second dose, and
wherein doses are administered every 6-8 days.

Several risk factors may be associated with the administration of TCR bispecific reagents, including cytokine release syndrome (CRS), local tumour inflammation, cytopenia and off target T cell activation. In some cases, dose limiting toxicities may arise at doses below a clinically effective dose. Furthermore, higher doses may result in off target recognition of normal tissues. The inventors have surprisingly found an intra-patient dose escalation regimen that allows IMC-F106C to be administered with a manageable safety profile and that demonstrates clinical activity.

The TCR-anti-CD3 fusion molecule for use in the invention comprises an anti-CD3 scFv covalently linked to the N-terminus of the beta chain of a TCR via a linker. This type of molecule is known as an ImmTAC® (Immune Mobilizing Monoclonal TCRs Against Cancer). ImmTAC® molecules are engineered to activate a potent T cell response to specifically kill target cancer cells. TCR-anti-CD3 fusion molecules for use in the invention (i.e. ImmTACs targeting PRAME) are described in WO/2018/234319, which is incorporated by reference herein in its entirety.

The terms “TCR-anti-CD3 fusion molecule”, “ImmTAC” and “T cell redirecting bispecific therapeutic agent” are used interchangeably herein.

The term “TCR beta chain-anti-CD3” used herein refers to the TCR beta chain portion of the ImmTAC together with the linker and the anti-CD3 scFv. The term “beta chain” is sometimes used in relation to ImmTACs as an alternative way to describe this portion of the molecule.

The sequences referred to herein are as follows:
SEQ ID NO: 1 HLA-A*02 restricted peptide: SLLQHLIGL
SEQ ID NO: 2 Amino acid sequence of the TCR alpha chain variable domain of the ImmTAC designated as IMC-F106C. CDRs (CDR1, CDR2 and CDR3) are underlined and are designated SEQ ID NO: 3, 4 and 5 respectively, framework regions (FR1, FR2, FR3 and FR4) are in italics and are designated SEQ ID NO: 27, 6, 7 and 28 respectively. Mutations with respect to native alpha chain are in bold.

GDAKTTQPNSMESNEEEPVHLPCNHSTISGTDYIHWYRQLPSQGPEY
VIHGLTSNVNNRMASLAIAEDRKSSTLILHRATLRDAAVYYCILILG
HSRLGNYIATFGKGTKLSVIP

SEQ ID NO: 8 Amino acid sequence of the TCR beta chain variable domain of the ImmTAC designated as IMC-F106C. CDRs (CDR1, CDR2 and CDR3) are underlined and are designated SEQ ID NO: 9, 10 and 11 respectively, framework regions (FR1, FR2, FR3 and FR4) are in italics and are designated SEQ ID NO: 29, 12, 13 and 30 respectively. Mutations with respect to native beta chain are in bold.

DGGITQSPKYLFRKEGQNVTLSCEQNLNHDAMYWYRQDPGQGLRLIY
YSQIMGDEQKGDIAEGYSVSREKKESFPLTVTSAQKNPTAFYLCASS
WWTGGASPIRFGPGTRLTVT

SEQ ID NO: 14 Amino acid sequence of the TCR alpha chain of the ImmTAC designated as IMC-F106C. CDRs (CDR1, CDR2 and CDR3) are underlined and are designated SEQ ID NO: 3, 4 and 5 respectively, framework regions (FR1, FR2, FR3 and FR4) are in italics and are designated SEQ ID NO: 27, 6, 7 and 28 respectively. The constant region is shown in bold and is designated SEQ ID NO: 15. Within the constant region, the non-native cysteine residue is double underlined (at position 48 of constant region).

GDAKTTQPNSMESNEEEPVHLPCNHSTISGTDYIHWYRQLPSQGPEY
VIHGLTSNVNNRMASLAIAEDRKSSTLILHRATLRDAAVYYCILILG
HSRLGNYIATFGKGTKLSVIPNIQNPDPAVYQLRDSKSSDKSVCLFT
DFDSQTNVSQSKDSDVYITDKCVLDMRSMDFKSNSAVAWSNKSDFA
CANAFNNSIIPEDT

SEQ ID NO: 16 Amino acid sequence of the TCR beta chain-anti-CD3 of the ImmTAC designated as IMC-F106C. Anti-CD3 scFv (amino acids 1-253) is shown in bold and underline and is designated SEQ ID NO: 17. The linker (GGGGS) appears immediately after the scFv, is shown in paler text and is designated SEQ ID NO: 18. CDRs (CDR1, CDR2 and CDR3) are underlined and are designated SEQ ID NO: 9, 10 and 11 respectively, framework regions (FR1, FR2, FR3 and FR4) are in italics and are designated SEQ ID NO: 29, 12, 13 and 30 respectively. Constant region is shown in bold (no underline) and is designated SEQ ID NO: 19. Within the constant region, the non-native cysteine residue is double underlined (at position 57 of constant region). Additional non-native amino acids at position 75 and position 89 of the constant region are also double underlined.

AIQMTQSPSSLSASVGDRVTITCRASQDIRNYLNWYQQKPGKAPKLL
IYYTSRLESGVPSRFSGSGSGTDYTLTISSLQPEDFATYYCQQGNTL
PWTFGQGTKVEIKGGGGSGGGGSGGGGSGGGGSGGGSEVQLVESGGG
LVQPGGSLRLSCAASGYSFTGYTMNWVRQAPGKGLEWVALINPYKGV
STYNQKFKDRFTISVDKSKNTAYLQMNSLRAEDTAVYYCARSGYYGD
SDWYFDVWGQGTLVTVSSGGGGSDGGITQSPKYLFRKEGQNVTLSCE
QNLNHDAMYWYRQDPGQGLRLIYYSQIMGDEQKGDIAEGYSVSREKK
ESFPLTVTSAQKNPTAFYLCASSWWTGGASPIRFGPGTRLTVTEDLK
NVFPPEVAVFEPSEAEISHTQKATLVCLATGFYPDHVELSWWVNGKE
VHSGVCTDPQPLKEQPALNDSRYALSSRLRVSATFWQDPRNHFRCQV
QFYGLSENDEWTQDRAKPVTQIVSAEAWGRAD

### CDR Sequences of the ImmTAC Designated as IMC-F106C:

| Alpha | Alpha | Alpha | Beta | Beta | Beta |
|---|---|---|---|---|---|
| chain | chain | chain | chain | chain | chain |
| CDR1 | CDR2 | CDR3 | CDR1 | CDR2 | CDR3 |
| TISGTDY | GLTSN | CILILGH | LNHDA | SQIMGDE | CASSWWT |
| (SEQ ID | (SEQ ID | SRLGNYI | (SEQ ID | (SEQ ID | GGASPIR |
| NO: 3) | NO: 4) | ATF | NO: 9) | NO: 10) | F |
| | | (SEQ ID | | | (SEQ ID |
| | | NO: 5) | | | NO: 11) |

### Framework Region Sequences of the ImmTAC Designated as IMC-F106C:

| Alpha | Alpha | Alpha | Alpha |
|---|---|---|---|
| chain | chain | chain | chain |
| FR1 | FR2 | FR3 | FR4 |
| GDAKTTQ | IHWYRQL | VNNRMAS | GKGTKLS |
| PNSMESN | PSQGPEY | LAIAEDR | VIP |
| EEEPVHL | VIH | KSSTLIL | (SEQ ID |
| PCNHS | (SEQ ID | HRATLRD | NO: 28) |
| (SEQ ID | NO: 6) | AAVYY | |
| NO: 27) | | (SEQ ID | |
| | | NO: 7) | |
| Beta | Beta | Beta | Beta |
| chain | chain | chain | chain |
| FR1 | FR2 | FR3 | FR4 |
| DGGITQS | MYWYRQD | QKGDIAE | GPGTRLT |
| PKYLFRK | PGQGLRL | GYSVSRE | VT |
| EGQNVTL | IYY | KKESFPL | (SEQ ID |
| SCEQN | (SEQ ID | TVTSAQK | NO: 30) |
| (SEQ ID | NO: 12) | NPTAFYL | |
| NO: 29) | | (SEQ ID | |
| | | NO: 13) | |

### Additional Linker Sequences Referred to Herein:

(SEQ ID NO: 20)
GGGSG,
(SEQ ID NO: 21)
GGSGG,
(SEQ ID NO: 22)
GSGGG,
(SEQ ID NO: 23)
GSGGGP,
(SEQ ID NO: 24)
GGEPS,
(SEQ ID NO: 25)
GGEGGGP,
and
(SEQ ID NO: 26)
GGEGGGSEGGGS

The TCR-anti-CD3 fusion molecule for use in the invention comprises:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively.

In other words, although the molecule may have some variation in the TCR alpha chain amino acid sequence compared to the sequence of SEQ ID NO: 14 (as long as the TCR alpha chain amino acid sequence has at least 90% identity to SEQ ID NO: 14) and/or some variation in the TCR beta chain-anti-CD3 amino acid sequence compared to the sequence of SEQ ID NO: 16 (as long as the TCR beta chain-anti-CD3 amino acid sequence has at least 90% identity to the amino acid sequence of SEQ ID NO: 16), the CDRs of the TCR alpha chain must have the amino acid sequences of SEQ ID NOs: 3, 4 and 5 respectively and the CDRs of TCR beta chain must have the amino acid sequences of SEQ ID NOs: 9, 10 and 11 respectively. The requirement for the TCR alpha chain variable domain to comprise CDRs 1, 2 and 3 having the amino acid sequences of SEQ ID NOs: 3, 4 and 5 respectively and the requirement for the TCR beta chain variable domain to comprise CDRs 1, 2 and 3 having the amino acid sequences of SEQ ID NOS: 9, 10 and 11 respectively thus applies to all aspects and embodiments of the invention described herein. The TCR alpha chain variable domain thus comprises CDRs 1, 2 and 3 having 100% identity to the amino acid sequences of SEQ ID NOs: 3, 4 and 5 respectively and the TCR beta chain variable domain comprises CDRs 1, 2 and 3 having 100% identity to the amino acid sequences of SEQ ID NOs: 9, 10 and 11 respectively.

Within the scope of the invention are phenotypically silent variants of the TCR-anti-CD3 fusion molecule designated as IMC-F106C, which has a TCR alpha chain amino acid sequence corresponding to SEQ ID NO: 14 and a TCR beta chain-anti-CD3 amino acid sequence corresponding to SEQ ID NO: 16. As used herein the term “phenotypically silent variant” is understood to refer to a TCR-anti-CD3 fusion molecule which incorporates one or more further amino acid changes, including substitutions, insertions and deletions, compared to the sequences of SEQ ID NO: 14 and SEQ ID NO: 16 and which TCR-anti-CD3 fusion molecule has a similar phenotype to or the same phenotype as the TCR-anti-CD3 fusion molecule designated as IMC-F106C. For the purposes of this application, TCR-anti-CD3 fusion molecule phenotype comprises antigen binding affinity (K_{D} and/or binding half-life) and antigen specificity. A phenotypically silent variant may have a K_{D} and/or binding half-life for the SLLQHLIGL (SEQ ID NO: 1) HLA-A*02 complex within 50%, or more preferably within 20%, of the measured Kp and/or binding half-life of the TCR-anti-CD3 fusion molecule designated as IMC-F106C, when measured under identical conditions (for example at 25° C. and/or on the same SPR chip). Suitable conditions are further provided in Example 3 of WO/2018/234319, which is incorporated herein by reference. Antigen specificity is further defined below. As is known to those skilled in the art, it may be possible to produce TCRs that incorporate changes in the variable domains thereof compared to those detailed above without altering the affinity of the interaction with the SLLQHLIGL (SEQ ID NO: 1) HLA-A*02 complex. In particular, such silent mutations may be incorporated within parts of the sequence that are known not to be directly involved in antigen binding. Such trivial variants are included in the scope of this invention.

Phenotypically silent variants may contain one or more conservative substitutions and/or one or more tolerated substitutions. Tolerated and conservative substitutions may result in a change in the Kp and/or binding half-life for the SLLQHLIGL (SEQ ID NO: 1) HLA-A*02 complex within 50%, or more preferably within 20%, even more preferably within 10%, of the measured Kp and/or binding half-life of the TCR-anti-CD3 fusion molecule designated as IMC-F106C, when measured under identical conditions (for example at 25° C. and/or the same SPR chip), provided that the change in Kp does not result in the affinity being less than (i.e. weaker than) 200 μm. By tolerated substitutions it is meant those substitutions which do not fall under the definition of conservative as provided below but are nonetheless phenotypically silent.

The TCR-anti-CD3 fusion molecule for use in the present invention may include one or more conservative substitutions which have a similar amino acid sequence and/or which retain the same function (i.e. are phenotypically silent as defined above). The skilled person is aware that various amino acids have similar properties and thus substitutions between them are “conservative”. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of this nature are often referred to as “conservative” or “semi-conservative” amino acid substitutions. The present invention therefore extends to use of a TCR-anti-CD3 fusion molecule comprising an amino acid sequence described above but with one or more conservative substitutions and/or one or more tolerated substitutions in the sequence, such that the TCR alpha chain amino acid sequence has at least 90% identity (such as 91%, 92%, 93%, 94%, 95%, 96%, 97%, 98% or 99% identity) to the amino acid sequence of SEQ ID NO: 14, and the TCR beta chain-anti-CD3 amino acid sequence has at least 90% identity (such as 91%, 92%, 93%, 94%, 95%, 96%, 97%, 98% or 99% identity) to the amino acid sequence of SEQ ID NO: 16, and provided that the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules. BLAST protein searches can be performed with the XBLAST program, score=50, wordlength=3 to obtain amino acid sequences homologous to protein molecules for use in the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

Mutations, including conservation and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning-A Laboratory Manual (3rd Ed.) CSHL Press. Further information on ligation independent cloning (LIC) procedures can be found in Rashtchian, (1995) Curr Opin Biotechnol 6 (1): 30-6. The TCR sequences provided by the invention may be obtained from solid state synthesis, or any other appropriate method known in the art.

The TCR-anti-CD3 fusion molecules for use in the present invention have the property of binding the SLLQHLIGL (SEQ ID NO: 1) HLA-A*02 complex. TCR-anti-CD3 fusion molecules for use in the present invention have been found to strongly recognise this epitope relative to other, irrelevant epitopes, and are thus particularly suitable as targeting vectors for delivery of therapeutic agents or detectable labels to cells and tissues displaying those epitopes. Specificity in the context of TCR-anti-CD3 fusion molecule for use in the present invention relates to their ability to recognise HLA-A*02 target cells that are antigen positive, whilst having minimal ability to recognise HLA-A*02 target cells that are antigen negative.

Specificity can be measured in vitro, for example in cellular assays such as those described in Example 6 of WO/2018/234319, which is incorporated herein by reference. Recognition may be determined by measuring the level of T cell activation in the presence of a TCR-anti-CD3 fusion molecule for use in the invention and target cells. Minimal recognition of antigen negative target cells is defined as a level of T cell activation of less than 20%, preferably less than 10%, preferably less than 5%, and more preferably less than 1%, of the level produced in the presence of antigen positive target cells, when measured under the same conditions and at a therapeutically relevant concentration. For TCR-anti-CD3 fusion molecules for use in the invention, a therapeutically relevant concentration may be defined as below 10 nM, for example below 1 nM or below 100 pM. Antigen positive cells may be obtained by peptide-pulsing using a suitable peptide concentration to obtain a level of antigen presentation comparable to cancer cells (for example, 10-9 M peptide, as described in Bossi et al., (2013) Oncoimmunol. 1; 2 (11): e26840) or, they may naturally present said peptide. Preferably, both antigen positive and antigen negative cells are human cells. Preferably antigen positive cells are human cancer cells. Antigen negative cells preferably include those derived from healthy human tissues.

Specificity may additionally, or alternatively, relate to the ability of a TCR-anti-CD3 fusion molecule to bind to SLLQHLIGL (SEQ ID NO: 1) HLA-A*02 complex and not to a panel of alternative peptide-HLA complexes. This may, for example, be determined by the Biacore method of Example 3 of WO/2018/234319, which is incorporated herein by reference. Said panel may contain at least 5, and preferably at least 10, alternative peptide-HLA-A*02 complexes. The alternative peptides may share a low level of sequence identity with SEQ ID NO: 1 and may be naturally presented. Alternative peptides may be derived from proteins expressed in healthy human tissues. Binding to SLLQHLIGL-HLA-A*02 complex may be at least 2 fold greater than to other naturally-presented peptide HLA complexes, more preferably at least 10 fold, or at least 50 fold or at least 100 fold greater, even more preferably at least 400 fold greater.

An alternative or additional approach to determine TCR specificity may be to identify the peptide recognition motif of the TCR using sequential mutagenesis, e.g. alanine scanning. Residues that form part of the binding motif are those that are not permissible to substitution. Non permissible substitutions may be defined as those peptide positions in which the binding affinity of the TCR is reduced by at least 50%, or preferably at least 80% relative to the binding affinity for the non-mutated peptide. Such an approach is further described in Cameron et al., (2013), Sci Transl Med. 2013 Aug. 7; 5 (197): 197ra103 and WO2014096803. TCR specificity in this case may be determined by identifying alternative motif containing peptides, particularly alternative motif containing peptides in the human proteome, and testing these peptides for binding to the TCR. Binding of the TCR to one or more alternative peptides may indicate a lack of specificity. In this case further testing of TCR specificity via cellular assays may be required.

Preferred embodiments of TCR anti-CD3 fusion molecules for use in the invention comprise a TCR alpha chain amino acid sequence that has at least 91%, 92%, 93%, 94%, 95%, 96%, 97%, 98%, 99% or 100% identity to the amino acid sequence set forth in SEQ ID NO: 14, and a TCR beta chain-anti-CD3 amino acid sequence that has at least 91%, 92%, 93%, 94%, 95%, 96%, 97%, 98%, 99% or 100% identity, to the amino acid sequence set forth in SEQ ID NO: 16, as long as the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively. The TCR anti-CD3 fusion molecules for use in the invention may therefore vary in any region other than the CDRs.

For example, a TCR anti-CD3 fusion molecule for use in the invention may comprise a TCR alpha chain Framework 1 region (FR1) amino acid sequence that has at least 91%, 92%, 93%, 94%, 95%, 96%, 97%, 98%, 99% or 100% identity to the amino acid sequence set forth in SEQ ID NO: 27, and/or a TCR alpha chain Framework 2 region (FR2) amino acid sequence that has at least 91%, 92%, 93%, 94%, 95%, 96%, 97%, 98%, 99% or 100% identity to the amino acid sequence set forth in SEQ ID NO: 6, and/or a TCR alpha chain Framework 3 region (FR3) amino acid sequence that has at least 91%, 92%, 93%, 94%, 95%, 96%, 97%, 98%, 99% or 100% identity, to the amino acid sequence set forth in SEQ ID NO: 7 and/or a TCR alpha chain Framework 4 region (FR4) amino acid sequence that has at least 91%, 92%, 93%, 94%, 95%, 96%, 97%, 98%, 99% or 100% identity, to the amino acid sequence set forth in SEQ ID NO: 28. For example, the alpha chain FR1 and/or FR2 and/or FR3 and/or FR4 regions may each contain one or more, for example one, two or three, conservative substitutions and/or up to three tolerated substitutions.

A TCR anti-CD3 fusion molecule for use in the invention may alternatively or additionally comprise a TCR beta chain Framework 1 region (FR1) amino acid sequence that has at least 91%, 92%, 93%, 94%, 95%, 96%, 97%, 98%, 99% or 100% identity to the amino acid sequence set forth in SEQ ID NO: 29, and/or a TCR beta chain Framework 2 region (FR2) amino acid sequence that has at least 91%, 92%, 93%, 94%, 95%, 96%, 97%, 98%, 99% or 100% identity to the amino acid sequence set forth in SEQ ID NO: 12, and/or a TCR beta chain Framework 3 region (FR3) amino acid sequence that has at least 91%, 92%, 93%, 94%, 95%, 96%, 97%, 98%, 99% or 100% identity, to the amino acid sequence set forth in SEQ ID NO: 13 and/or a TCR beta chain Framework 4 region (FR4) amino acid sequence that has at least 91%, 92%, 93%, 94%, 95%, 96%, 97%, 98%, 99% or 100% identity, to the amino acid sequence set forth in SEQ ID NO: 30. For example, the beta chain FR1 and/or FR2 and/or FR3 and/or FR4 regions may each contain one or more, for example one, two or three, conservative substitutions and/or up to three tolerated substitutions.

A TCR anti-CD3 fusion molecule for use in the invention may alternatively or additionally comprise a TCR alpha chain variable domain amino acid sequence that has at least 91%, 92%, 93%, 94%, 95%, 96%, 97%, 98%, 99% or 100% identity to the amino acid sequence set forth in SEQ ID NO: 2, and/or a TCR beta chain variable domain amino acid sequence that has at least 91%, 92%, 93%, 94%, 95%, 96%, 97%, 98%, 99% or 100% identity, to the amino acid sequence set forth in SEQ ID NO: 8. For example, the TCR-anti-CD3 fusion molecule may comprise a TCR alpha chain variable domain having the amino acid sequence of SEQ ID NO: 2 and a TCR beta chain variable domain having the amino acid sequence of SEQ ID NO: 8. Alternatively, the TCR-anti-CD3 fusion molecule may comprise a TCR alpha chain variable domain having one or more mutations, for example one, two or three, conservative substitutions and/or up to three tolerated substitutions, compared to the amino acid sequence of SEQ ID NO: 2 and/or a TCR beta chain variable domain having one or more mutations, for example one, two or three, conservative substitutions and/or up to three tolerated substitutions, compared to the amino acid sequence of SEQ ID NO: 8.

A TCR anti-CD3 fusion molecule for use in the invention may alternatively or additionally comprise a TCR alpha chain constant region amino acid sequence that has at least 91%, 92%, 93%, 94%, 95%, 96%, 97%, 98%, 99% or 100% identity to the amino acid sequence set forth in SEQ ID NO: 15, and/or a TCR beta chain constant region amino acid sequence that has at least 91%, 92%, 93%, 94%, 95%, 96%, 97%, 98%, 99% or 100% identity, to the amino acid sequence set forth in SEQ ID NO: 19. For example, the TCR alpha chain constant region may have one, two or three, conservative substitutions and/or up to three tolerated substitutions, compared to the amino acid sequence of SEQ ID NO: 15. For example, the TCR beta chain constant region may have one, two or three, conservative substitutions and/or up to three tolerated substitutions, compared to the amino acid sequence of SEQ ID NO: 19.

Alternatively or additionally, the anti-CD3 scFv in the TCR anti-CD3 fusion molecule for use in the invention may comprise an amino acid sequence that has at least 91%, 92%, 93%, 94%, 95%, 96%, 97%, 98%, 99% or 100% identity to the amino acid sequence set forth in SEQ ID NO: 17.

In the TCR anti-CD3 fusion molecule for use in the invention, the TCR beta chain is linked to the anti-CD3 antibody sequence via a linker. The amino acid sequence of the linker may be selected from the group consisting of GGGGS (SEQ ID NO: 18), GGGSG (SEQ ID NO: 20), GGSGG (SEQ ID NO: 21), GSGGG (SEQ ID NO: 22), GSGGGP (SEQ ID NO: 23), GGEPS (SEQ ID NO: 24), GGEGGGP (SEQ ID NO: 25), and GGEGGGSEGGGS (SEQ ID NO: 26). Typically, the linker sequence is GGGGS (SEQ ID NO: 18). Alternatively, the linker may have one or more mutations, for example one, two or three, conservative substitutions and/or up to three tolerated substitutions compared to any of the linker sequences of SEQ ID NOs: 18 and 20-26.

It can therefore be seen that any of these embodiments may be combined as long as the resulting TCR-anti-CD3 fusion molecule comprises a TCR alpha chain amino acid sequence that has at least 90% identity (for example at least 91%, 92%, 93%, 94%, 95%, 96%, 97%, 98%, 99% or 100% identity) to the amino acid sequence of SEQ ID NO: 14, and a TCR beta chain-anti-CD3 amino acid sequence that has at least 90% identity (for example at least 91%, 92%, 93%, 94%, 95%, 96%, 97%, 98%, 99% or 100% identity) to the amino acid sequence of SEQ ID NO: 16, and the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively.

The TCR-anti-CD3 fusion molecule for use in the invention may comprise a TCR alpha chain having an amino acid sequence corresponding to SEQ ID NO: 14 and a TCR beta chain-anti-CD3 amino acid sequence corresponding to SEQ ID NO: 16. These are the TCR alpha chain amino acid sequence and the TCR beta chain-anti-CD3 amino acid sequence respectively of IMC-F106C.

The alpha chain constant region having the amino acid sequence of SEQ ID NO: 15 includes a modification relative to the corresponding native/naturally occurring alpha chain whereby amino acid T48 of the constant region is replaced with C48, as shown herein.

The beta chain constant region having the amino acid sequence of SEQ ID NO: 19 also includes a modification relative to the native/naturally occurring beta chain whereby S57 is replaced with C57, as shown in herein. These cysteine substitutions relative to the native alpha and beta chain constant chain sequences enable the formation of a non-native interchain disulphide bond which stabilises the refolded soluble TCR, i.e. the TCR formed by refolding extracellular alpha and beta chains (WO 03/020763). This non-native disulphide bond facilitates the display of correctly folded TCRs on phage (Li et al., Nat Biotechnol 2005 March; 23 (3): 349-54). In addition, the use of the stable disulphide linked soluble TCR enables more convenient assessment of binding affinity and binding half-life. The beta chain constant region having the amino acid sequence of SEQ ID NO: 19 also includes additional non-native amino acids at positions 75 (A75) and 89 (D89), as shown herein.

All of the sequences referred to in the present application are also referred to in WO 2018/234319, which is incorporated by reference herein. Table 1 shows how the parts of the ImmTAC molecule and the SEQ ID NOs referred to herein correspond to the SEQ ID NOs of WO 2018/234319.

TABLE 1

| | SEQ ID NO | SEQ ID NO | |
|---|---|---|---|
| | referred to | referred to in | |
| Description of sequence | herein | WO 2018/234319 | Additional notes |
| HLA-A*02 restricted | SEQ ID NO: 1 | SEQ ID NO: 1 | |
| peptide derived from | | | |
| germline cancer antigen | | | |
| PRAME | | | |
| TCR alpha chain variable | SEQ ID NO: 2 | SEQ ID NO: 7 | Referred to as |
| domain of the ImmTAC | | | a79 in WO |
| designated as IMC-F106C | | | 2018/234319 |
| TCR alpha chain CDR1 of | SEQ ID NO: 3 | — | |
| IMC-F106C | | | |
| TCR alpha chain CDR2 of | SEQ ID NO: 4 | — | |
| IMC-F106C | | | |
| TCR alpha chain CDR3 of | SEQ ID NO: 5 | — | |
| IMC-F106C | | | |
| TCR alpha chain FR1 of | SEQ ID NO: 27 | | |
| IMC-F106C | | | |
| TCR alpha chain FR2 of | SEQ ID NO: 6 | — | |
| IMC-F106C | | | |
| TCR alpha chain FR3 of | SEQ ID NO: 7 | — | |
| IMC-F106C | | | |
| TCR alpha chain FR4 of | SEQ ID NO: 28 | | |
| IMC-F106C | | | |
| TCR beta chain variable | SEQ ID NO: 8 | SEQ ID NO: 17 | Referred to as |
| domain of the ImmTAC | | | b74 in WO |
| designated as IMC-F106C | | | 2018/234319 |
| TCR beta chain CDR1 of | SEQ ID NO: 9 | — | |
| IMC-F106C | | | |
| TCR beta chain CDR2 of | SEQ ID NO: 10 | — | |
| IMC-F106C | | | |
| TCR beta chain CDR3 of | SEQ ID NO: 11 | — | |
| IMC-F106C | | | |
| TCR beta chain FR1 of | SEQ ID NO: 29 | | |
| IMC-F106C | | | |
| TCR beta chain FR2 of | SEQ ID NO: 12 | — | |
| IMC-F106C | | | |
| TCR beta chain FR3 of | SEQ ID NO: 13 | — | |
| IMC-F106C | | | |
| TCR beta chain FR4 of | SEQ ID NO: 30 | | |
| IMC-F106C | | | |
| TCR alpha chain of the | SEQ ID NO: 14 | SEQ ID NO: 27 | Referred to as |
| ImmTAC designated as | | | a79 in WO |
| IMC-F106C | | | 2018/234319 |
| TCR alpha chain constant | SEQ ID NO: 15 | — | |
| region of IMC-F106C | | | |
| TCR beta chain-anti-CD3 | SEQ ID NO: 16 | SEQ ID NO: 28 | Referred to as |
| portion of the ImmTAC | | | b74 in WO |
| designated as IMC-F106C | | | 2018/234319 |
| Anti-CD3 scFv portion of | SEQ ID NO: 17 | — | Corresponds to |
| IMC-F106C | | | amino acids 1- |
| | | | 253 of SEQ ID |
| | | | NO: 16 of present |
| | | | application |
| Linker of IMC-F106C | SEQ ID NO: 18 | SEQ ID NO: 31 | |
| TCR beta chain constant | SEQ ID NO: 19 | — | |
| region of IMC-F106C | | | |
| Alternative linker | SEQ ID NO: 20 | SEQ ID NO: 32 | |
| sequence | | | |
| Alternative linker | SEQ ID NO: 21 | SEQ ID NO: 33 | |
| sequence | | | |
| Alternative linker | SEQ ID NO: 22 | SEQ ID NO: 34 | |
| sequence | | | |
| Alternative linker | SEQ ID NO: 23 | SEQ ID NO: 35 | |
| sequence | | | |
| Alternative linker | SEQ ID NO: 24 | SEQ ID NO: 36 | |
| sequence | | | |
| Alternative linker | SEQ ID NO: 25 | SEQ ID NO: 37 | |
| sequence | | | |
| Alternative linker | SEQ ID NO: 26 | SEQ ID NO: 38 | |
| sequence | | | |

The ImmTAC designated as IMC-F106C and which is described in the present application is designated ImmTAC2 in WO 2018/234319.

In this aspect of the present invention, the TCR-anti-CD3 fusion molecule is administered as follows:
(a) at least one first dose in the range of from 5-40 μg;
(b) at least one second dose in the range of from 15-80 μg; and then
(c) at least one third dose in the range of from 60-400 μg,
wherein the second dose is higher than the first dose and the third dose is higher than the second dose, and
wherein doses are administered every 6-8 days.

The dosage regimen of the present invention is a dose escalation regimen, in which increasing doses of the TCR-anti-CD3 fusion molecule are sequentially administered. Doses are thus administered in the specified order: first dose, then second dose, then third dose. By “first dose” is meant a dose of the TCR-anti-CD3 fusion molecule at a first level within the specified range. By “second dose” is meant a dose of the TCR-anti-CD3 fusion molecule at a second level within the specified range, which is higher than the first level. By “third dose” is meant a dose of the TCR-anti-CD3 fusion molecule at a third level within the specified range, which is higher than the second level. It will be appreciated that according to the dosage regimen of the present invention, a patient may receive more than three doses of the TCR-anti-CD3 fusion molecule, because more than one first dose, more than one second dose and/or more than one third dose may be administered.

In the present invention, the respective doses are expressed as a specified weight of therapeutic irrespective of the patient's weight or whether the same amount of therapeutic would be administered if calculated through one of the other methods routinely used to calculate an appropriate dosage for a patient, such as weight of therapeutic per kg of body weight, body surface area or lean muscle mass etc. The specified weight of therapeutic is typically administered at weekly intervals, e.g. on days 1, 8, 15, 22, etc of the treatment regimen, but the dosing interval could be longer or shorter. Thus, doses (each dose, i.e. at least one first dose, at least one second dose and at least one third dose) are administered every 6-8 days. Preferably, doses (i.e. at least one first dose, at least one second dose and at least one third dose) are administered every 7 days. The respective doses may be separated by different intervals. Alternatively, they may be separated by the same interval.

The first dose is in the range of from 5-40 μg. It may be in the range of from 6-40 μg, 5-10 μg, 10-20 μg or 10-30 μg. Preferably, the first dose is in the range of from 10-30 μg. The dose may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 μg. Preferred first doses include 6, 15 or 20 μg, which may be administered on a weekly basis. Preferably, the first dose is 20 μg. One first dose may be administered. Alternatively, more than one first dose, for example 2-5 first doses, such as two first doses, may be administered. This may be required, for example, if the patient experiences an adverse event (AE) following administration of the first dose. In this case, it may be preferred to administer one or more additional first doses before escalating to the second dose. It is preferred that, if two or more first doses are administered, they are the same. However, they may be different.

The second dose is in the range of from 15-80 μg. It may be in the range of from 20-80 μg, 15-30 μg, 30-50 μg, 40-70 μg or 50-70 μg. Preferably, the second dose is in the range of from 40-70 μg. The second dose may be 15, 20, 25, 30, 35, 40, 45, 50, 55, 60, 65, 70, 75 or 80 μg. Preferred second doses include 20, 40, 60 or 80 μg, which may be administered on a weekly basis. Preferably, the second dose is 60 μg. One second dose may be administered. Alternatively, more than one second dose, for example 2-5 second doses, such as two second doses, may be administered. Again, this may be required, for example, if the patient experiences an adverse event following administration of the second dose. In this case, it may be preferred to administer one or more additional first doses before escalating to the second dose. It is preferred that, if two or more second doses are administered, they are the same. However, they may be different.

Preferably, the first dose is in the range of from 10-30 μg and the second dose is in the range of from 40-70 μg.

The third dose is in the range of from 60-400 μg. It may be in the range of from 80-400 μg, 70-250 μg, 60-100 μg, 100-200 μg, 150-300 μg, 70-90 μg, 140-180 μg or 220-260 μg. For example, it may be in the range of from 80-240 μg, 150-400 μg, 150-330 μg, 160-320μ, 200-320μ, 240-320μ, 150-170μ, 190-210μ, 230- 250μ, 250-270μ, 290-310 μg or 310-330 μg. The third dose may be at least 150 μg. The third dose may be 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 or 400 μg. One preferred third dose is 80 μg. Another preferred third dose is 160 μg. Further preferred third doses include 200 μg, 240 μg, 260 μg, 300 μg and 320 μg. One or more third doses may be administered. Typically, multiple third doses are administered, for example, every 6-8 days, preferably every 7 days, until treatment is stopped. Treatment may be continued for one or more months or one or more years. This third dose that is continued can be referred to as, and correspond to, the “maintenance dose”.

Treatment may be stopped, for example due to unacceptable toxicity or because the patient has shown an unacceptable level of disease progression. Alternatively, treatment may be stopped, for example, because the patient's symptoms have reduced in severity and/or tumour has shrunk to a level at which treatment with the TCR-anti-CD3 fusion molecule is deemed no longer necessary. The decision on whether and when to stop treatment can be determined by a clinician. The same dose may be used subsequently. Alternatively, the dose may be escalated. For example, the dose may be 5, 10, 15, 20, 30, 40 or 50 μg higher.

Any combination of ranges of first, second and third doses as described herein are contemplated. For example, the first dose may be in the range of 6-40 μg, the second dose may be in the range of 20-80 μg and the third dose may be in the range of 80-400 μg. The first dose may be in the range of from 5-10 μg, 10-20 μg or 10-30 μg, the second dose may be in the range of from 15-30 μg, 30-50 μg or 50-70 μg and the third dose may be in the range of from 60-100 μg, 100-200 μg, 150-300 μg, 150-400 μg, 150-330 μg, 160-320μ, 150-170μ, 190-210μ, 230-250μ, 250-270μ, 290-310μ g or 310-330 μg, in any combination, provided the second dose is higher than the first dose and the third dose is higher than the second dose.

The first dose may be 6 μg, the second dose may be 20 μg and the third dose may be in the range of from 80-120 μg. The first dose may be 6 μg, the second dose may be 20 μg and the third dose may be 80 μg, 100 μg or 120 μg. In other words, the first dose may be 6 μg, the second dose may be 20 μg and the third dose may be 80 μg. The first dose may be 6 μg, the second dose may be 20 μg and the third dose may be 100 μg. The first dose may be 6 μg, the second dose may be 20 μg and the third dose may be 120 μg.

The first dose may be 15 μg, the second dose may be 40 μg and the third dose may be in the range of from 140-180 μg. The first dose may be 15 μg, the second dose may be 40 μg and the third dose may be 140 μg, 160 μg or 180 μg. In other words, the first dose may be 15 μg, the second dose may be 40 μg and the third dose may be 140 μg. The first dose may be 15 μg, the second dose may be 40 μg and the third dose may be 160 μg. The first dose may be 15 μg, the second dose may be 40 μg and the third dose may be 180 μg.

The first dose may be 20 μg, the second dose may be 60 μg and the third dose may be at least 150 μg. The first dose may be 20 μg, the second dose may be 60 μg and the third dose may be in the range of from 150-400 μg, for example, from 150-330 μg, from 150-170 μg, from 160-320 μg, from 190-210 μg, from 220-320 μg, from 240-320 μg, from 230-250 μg, from 250-270 μg, from 220-260 μg, from 290-310 μg, or from 310-330 μg.

The first dose may be 20 μg, the second dose may be 60 μg and the third dose may be 160 μg, 200 μg, 220 μg, 240 μg, 260 μg, 300 μg or 320 μg. The first dose may be 20 μg, the second dose may be 60 μg and the third dose may be 160 μg. The first dose may be 20 μg, the second dose may be 60 μg and the third dose may be 200 μg. The first dose may be 20 μg, the second dose may be 60 μg and the third dose may be 220 μg. The first dose may be 20 μg, the second dose may be 60 μg and the third dose may be 240 μg. The first dose may be 20 μg, the second dose may be 60 μg and the third dose may be 260 μg. The first dose may be 20 μg, the second dose may be 60 μg and the third dose may be 300 μg. The first dose may be 20 μg, the second dose may be 60 μg and the third dose may be 320 μg.

The first dose may be in the range of from 10-30 μg, the second dose may be in the range of from 40-70 μg and the third dose may be at least 150 μg. The first dose may be in the range of from 10-30 μg, the second dose may be in the range of from 40-70 μg and the third dose may be in the range of from 150-400 μg. The first dose may be in the range of from 10-30 μg, the second dose may be in the range of from 40-70 μg and the third dose may be in the range of from 150-330 μg.

The first dose may be in the range of from 10-30 μg, the second dose may be in the range of from 40-70 μg and the third dose may be in the range of from 150-170 μg. The first dose may be in the range of from 10-30 μg, the second dose may be in the range of from 40-70 μg and the third dose may be 160 μg.

The first dose may be in the range of from 10-30 μg, the second dose may be in the range of from 40-70 μg and the third dose may be in the range of from 190-210 μg. The first dose may be in the range of from 10-30 μg, the second dose may be in the range of from 40-70 μg and the third dose may be 200 μg.

The first dose may be in the range of from 10-30 μg, the second dose may be in the range of from 40-70 μg and the third dose may be in the range of from 230-250 μg. The first dose may be in the range of from 10-30 μg, the second dose may be in the range of from 40-70 μg and the third dose may be 240 μg.

The first dose may be in the range of from 10-30 μg, the second dose may be in the range of from 40-70 μg and the third dose may be in the range of from 250-270 μg. The first dose may be in the range of from 10-30 μg, the second dose may be in the range of from 40-70 μg and the third dose may be 260 μg.

The first dose may be in the range of from 10-30 μg, the second dose may be in the range of from 40-70 μg and the third dose may be in the range of from 290-310 μg. The first dose may be in the range of from 10-30 μg, the second dose may be in the range of from 40-70 μg and the third dose may be 300 μg.

The first dose may be in the range of from 10-30 μg, the second dose may be in the range of from 40-70 μg and the third dose may be in the range of from 310-330 μg. The first dose may be in the range of from 10-30 μg, the second dose may be in the range of from 40-70 μg and the third dose may be 320 μg.

In a second aspect, the invention provides a TCR-anti-CD3 fusion molecule comprising:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating PRAME positive cancer in a patient comprising administering the TCR-anti-CD3 fusion molecule to said patient intravenously, wherein the method comprises administration of:
(a) at least one first dose;
(b) at least one second dose; and then
(c) at least one third dose in the range of from 60-400 μg,
wherein the second dose is higher than the first dose and the third dose is higher than the second dose, and
wherein doses are administered every 6-8 days.

This aspect of the invention relates to a dose escalation dosage regimen in which at least one third dose in the range of from 60-400 μg is administered following a dose escalation. Doses (each dose, i.e. at least one first dose, at least one second dose and at least one third dose) are administered every 6-8 days. Preferably, doses (i.e. at least one first dose, at least one second dose and at least one third dose) are administered every 7 days. The respective doses may be separated by different intervals. Alternatively, they may be separated by the same interval.

The first and second dose may be determined by a clinician, or may be as defined herein in relation to the first aspect of the invention.

Suitable third doses for the second aspect of the invention are described above in relation to the first aspect of the invention. The third dose is in the range of from 60-400 μg. It may be in the range of from 80-400μ, 70-250μ, 60-100μ, 100-200μ, 150-300μ, 70-90 μg, 140-180 μg or 220-260 μg. For example, it may be in the range of from 80-240 μg, 150-400 μg, 150-330 μg, 160-320 μg, 200-320 μg, 240-320 μg, 150-170 μg, 190-210 μg, 230-250 μg, 250-270 μg, 290-310 μg or 310-330 μg. The third dose may be at least 150 μg. The third dose may be 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 or 400 μg. One preferred third dose is 80 μg. Another is 160 μg. Further preferred third doses include 200 μg, 240 μg, 260 μg, 300 μg and 320 μg. One or more third doses may be administered. Typically, multiple third doses are administered, for example, every 6-8 days, preferably every 7 days, until treatment is stopped. Treatment may be continued for one or more months or one or more years. This third dose that is continued can be referred to as, and correspond to, the “maintenance dose”.

Treatment may be stopped, for example due to unacceptable toxicity or because the patient has shown an unacceptable level of disease progression. Alternatively, treatment may be stopped, for example, because the patient's symptoms have reduced in severity and/or tumour has shrunk to a level at which treatment with the TCR-anti-CD3 fusion molecule is deemed no longer necessary. The decision on whether and when to stop treatment can be determined by a clinician. The same dose may be used subsequently. Alternatively, the dose may be escalated. For example, the dose may be 5, 10, 15, 20, 30, 40 or 50 μg higher.

In the present invention, the TCR-anti-CD3 fusion molecule is administered intravenously (iv), typically by intravenous infusion.

The TCR-anti-CD3 fusion molecule for use in the present invention may be administered following a premedication regimen. As will be appreciated by a person of skill in the art, a premedication is the administration of medication prior to the administration of a treatment and is intended to counteract the potential side effects of the treatment. For example, the TCR-anti-CD3 fusion molecule may be administered following a steroid (corticosteroid) and/or non-steroid based premedication regimen. The steroid may be administered prior to administering the first, second and/or third dose, and is typically administered prior to administering the third dose. The steroid may be administered, for example, 15, 20, 25 or 30 minutes prior to administering the first, second and/or third dose. The steroid may be administered when the third dose is 140 μg or above (for example, when the third dose is 150, 160, 170, 180, 190, 200, 210, 220, 230, 240, 250, 260, 270, 280, 290, 300, 310, 320, 330, 340, 350, 360, 370, 380, 390 or 400 μg) and/or only prior to the third dose being administered for the first time.

The steroid may be dexamethasone. Dexamethasone may be administered intravenously and may be given at a dose in the range of 4-6 mg. Higher doses may be used, for example 8, 12, or 20 mg. Alternative steroids include prednisone, methylprednisolone and hydrocortisone.

Other premedications for use in combination with the dosage regimen of the invention include:
Paracetamol—typically administered orally at 1 g or equivalent
Ibuprofen—typically administered orally at 600-800 mg or equivalent
Diphenhydramine—typically administered orally at 50 mg.
Non-sedating antihistamines such as cetirizine (typically administered orally at 10 mg) or loratadine (typically administered orally at 10 mg).
An anti-emetic such as ondansetron (typically administered orally or intravenously at 8 mg).
Intravenous fluids—typically administered in the range of from 0.5-1 L. This is to mitigate the risk of hypotension, especially if the patient is experiencing dehydration, poor oral intake and/or nausea/vomiting.

Any such premedications may be used alone or in combination. The premedication(s) may be administered prior to administering the first, second and/or third dose, typically prior to administering the third dose. The premedication(s) may be administered, for example, 15, 20, 25 or 30 minutes prior to administering the first, second and/or third dose. If the patient develops an adverse event associated with any of these premedications, a reduced dose may be given. For example, a dose of 25 mg of diphenhydramine may be administered.

The TCR-anti-CD3 fusion molecule for use in the present invention may be administered as a monotherapy. Alternatively, it may be administered in combination with one or more anti-cancer therapies, preferably immuno-modulatory therapies or chemotherapy agents. Such anti-cancer therapies or chemotherapies include:
checkpoint inhibitors such as agents that target PD-1 or PD-L1, e.g. atezolizumab (TECENTRIQ®) (e.g. at 1200 mg every 3 weeks (Q3W)), pembrolizumab (e.g., at 400 mg every 6 weeks (Q6W), nivolumab, avelumab and durvalumab, and agents that target CTLA-4 such as ipilimumab and tremelimumab,
chemotherapy agents, such as dacarbazine and temozolamide,
immunotherapeutic agents, such as interleukin-2 (IL-2) and interferon (IFN)
BRAF inhibitors, such as vemurafenib and dabrafenib,
MEK inhibitors, such as trametinib,
TGF-β inhibitors such as galunisertib,
MET kinase inhibitors such as merestinib,
anti-angiogenic agents such as bevacizumab (Avastin®),
gemcitabine, e.g., at 1000 mg/m2 3 weeks on/1 week off, and
other TCR-anti-CD3 fusion molecules, such as tebentafusp.

The anti-cancer therapies or chemotherapies can be administered according to standard guidelines or recommendations, or manufacturer's prescribing information.

The TCR-anti-CD3 fusion molecule may be administered in combination with a checkpoint inhibitor. The checkpoint inhibitor may reduce immunosuppression within the tumour microenvironment, enhance the initial activity of IMC-F106C and/or prevent T-cell exhaustion, thereby sustaining the effectiveness of the emerging antitumour immune response. Also, the TCR-anti-CD3 fusion molecule may promote polyclonal T-cell recruitment into tumours, thereby overcoming a key resistance mechanism to checkpoint inhibitors.

The TCR-anti-CD3 fusion molecule for use according to the invention may be administered in combination with another TCR-anti-CD3 fusion molecule, i.e., a TCR-anti-CD3 molecule comprising a TCR that binds to a different peptide-MHC complex. The other fusion molecule may comprise a TCR that binds to a gp100 peptide-MHC complex. For example, the TCR-anti-CD3 fusion molecule for use according to the invention may be administered in combination with tebentafusp. Tebentafusp may be administered once weekly by intravenous infusion, according to its current prescribing information.

A preferred combination therapy uses atezolizumab in combination with a TCR-anti-CD3 fusion molecule as described herein. Atezolizumab is typically administered according to the current prescribing information, e.g., 840 mg every 2 weeks, 1200 mg every 3 weeks or 1680 mg every 4 weeks, by intravenous infusion.

Another preferred combination therapy uses pembrolizumab in combination with a TCR-anti-CD3 fusion molecule as described herein. Pembrolizumab is typically administered according to the current prescribing information, e.g., 200 mg every three weeks or 400 mg every six weeks, by intravenous infusion.

The TCR-anti-CD3 fusion molecule may be administered in combination with other therapeutic agents sequentially. The TCR-anti-CD3 fusion molecule may be administered on its own for the first and subsequent doses, with the additional therapeutic agents being added thereafter, or vice-versa. In embodiments of the present invention where a TCR-anti-CD3 fusion molecule and another anti-cancer therapy are administered in combination, the TCR-anti-CD3 fusion molecule may be administered alone in weeks 1 and 2 and the other anti-cancer therapy added in week 3 and subsequent weeks. For example, atezolizumab is typically administered once the patient has reached the third dose of the TCR-anti-CD3 fusion molecule. Typically, atezolizumab is administered prior to administration of the TCR-anti-CD3 fusion molecule. Administration of the TCR-anti-CD3 fusion molecule, typically by intravenous infusion, may commence, for example, 30 mins after infusion of atezolizumab.

Combination therapies may lead to an increased risk of immune-related toxicities, such as CRS. Accordingly, the dose of a TCR-anti-CD3 fusion molecule may be initially given as a single agent prior to combination dosing. Dosing of one or more additional anti-cancer therapies may be administered from week 3.

For administration to patients, the TCR-anti-CD3 fusion molecule may be provided as part of a pharmaceutical composition (e.g., sterile pharmaceutical composition) together with one or more pharmaceutically acceptable carriers or excipients. It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be in any suitable form for intravenous administration. Such compositions may be prepared by any method known in the art of pharmacy, for example by mixing the active ingredient with the carrier(s) or excipient(s) under sterile conditions.

The present invention relates to the treatment of PRAME positive cancers. By “PRAME positive cancer”, it is meant a cancer in which at least some of the cancer cells express PRAME. In other words, a PRAME positive cancer is a cancer associated with PRAME expression. The cancer may be known to be associated with expression of PRAME. For example, it may be known that the prevalence of PRAME expression is elevated in the cancer and thus PRAME expression may not be assessed, or may be assessed retrospectively. Alternatively, PRAME expression can be assessed using any method known in the art, including, for example, histological methods or other quantitative or qualitative measurements, including PCR, RNA expression analysis, and/or kits or sequence panels designed to measure the expression level of PRAME. However, the invention is not intended to be limited to the treatment of cancers for which PRAME expression can be detected by histological methods. In particular, the invention is not intended to be limited to the treatment of individual patients in whom PRAME expression can be detected, for example by histological methods. Rather, the invention is useful for the treatment of cancers and tumour types which are considered to be PRAME positive.

PRAME expression, when detected by histological methods like immunohistochemistry (IHC), can be quantified using an H-score. Expression of PRAME in individual cells or their sub-cellular compartments within a tumour are first detected and classified as either positive or negative. The positive cells can be further classified into high, medium, or low based on the IHC signal intensity. The H-score captures both the intensity and the proportion of the biomarker of interest from the IHC image and comprises values between 0 and 300, thereby offering a dynamic range to quantify abundance or a particular marker or gene.

Various cancers and tumour types are considered to be PRAME positive. PRAME positive cancers include, but are not limited to, melanoma, lung cancer, breast cancer, ovarian cancer, endometrial cancer, oesophageal cancer, bladder cancer, head and neck cancer, uterine cancer, Acute myeloid leukemia, chronic myeloid leukemia, and Hodgkin's lymphoma. For example, the PRAME positive cancer may be melanoma. The melanoma may be uveal melanoma or cutaneous melanoma. The lung cancer may be non-small cell lung carcinoma (NSCLC) or small cell lung cancer (SCLC). The breast cancer may be triple-negative breast cancer (TNBC) The bladder cancer may be urothelial carcinoma. The oesophageal cancer may be gastroesophageal junction (GEJ) adenocarcinoma. The ovarian cancer may be epithelial ovarian cancer, such as high grade serous ovarian cancer. The cancer may have relapsed from, be refractory to, or be intolerant of standard treatment regimens.

The first aspect of the invention extends to a TCR-anti-CD3 fusion molecule comprising:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating cancer in a patient comprising administering the TCR-anti-CD3 fusion molecule to said patient intravenously, wherein the method comprises administration of:
(a) at least one first dose in the range of from 5-40 μg;
(b) at least one second dose in the range of from 15-80 μg; and then
(c) at least one third dose in the range of from 60-400 μg,
wherein the second dose is higher than the first dose and the third dose is higher than the second dose, and
wherein doses are administered every 6-8 days.

The second aspect of the invention extends to a TCR-anti-CD3 fusion molecule comprising:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating cancer in a patient comprising administering the TCR-anti-CD3 fusion molecule to said patient intravenously, wherein the method comprises administration of:
(a) at least one first dose;
(b) at least one second dose; and then
(c) at least one third dose in the range of from 60-400 μg,
wherein the second dose is higher than the first dose and the third dose is higher than the second dose, and
wherein doses are administered every 6-8 days.

In both of these aspects, the cancer may be selected from the group consisting of melanoma, lung cancer, breast cancer, ovarian cancer, endometrial cancer, oesophageal cancer, bladder cancer, head and neck cancer, uterine cancer, Acute myeloid leukemia, chronic myeloid leukemia, and Hodgkin's lymphoma. For example, the cancer may be melanoma. The melanoma may be uveal melanoma or cutaneous melanoma. The lung cancer may be non-small cell lung carcinoma (NSCLC) or small cell lung cancer (SCLC). The breast cancer may be triple-negative breast cancer (TNBC) The bladder cancer may be urothelial carcinoma. The oesophageal cancer may be gastroesophageal junction (GEJ) adenocarcinoma. The ovarian cancer may be epithelial ovarian cancer, such as high grade serous ovarian cancer.

The first aspect of the invention also extends to the use of a TCR anti-CD3 fusion molecule in the manufacture of a medicament for the treatment of PRAME positive cancer by intravenous administration of a TCR-anti-CD3 fusion molecule as defined herein, wherein the treatment of PRAME positive cancer comprises administration of:
(a) at least one first dose in the range of from 5-40 μg;
(b) at least one second dose in the range of from 15-80 μg; and then
(c) at least one third dose in the range of from 60-400 μg,
wherein the second dose is higher than the first dose and the third dose is higher than the second dose, and
wherein doses are administered every 6-8 days.

The second aspect of the invention also extends to the use of a TCR anti-CD3 fusion molecule in the manufacture of a medicament for the treatment of PRAME positive cancer by intravenous administration of a TCR-anti-CD3 fusion molecule as defined herein, wherein the treatment of PRAME positive cancer comprises administration of:
(a) at least one first dose;
(b) at least one second dose; and then
(c) at least one third dose in the range of from 60-400 μg,
wherein the second dose is higher than the first dose and the third dose is higher than the second dose, and
wherein doses are administered every 6-8 days.

The first aspect of the invention also extends to a method of treating PRAME positive cancer in a patient comprising administering a TCR-anti-CD3 fusion molecule to said patient intravenously, wherein the TCR-anti-CD3 fusion molecule comprises:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively, wherein the method comprises administration of:
(a) at least one first dose in the range of from 5-40 μg;
(b) at least one second dose in the range of from 15-80 μg; and then
(c) at least one third dose in the range of from 60-400 μg,
wherein the second dose is higher than the first dose and the third dose is higher than the second dose, and
wherein doses are administered every 6-8 days.

The second aspect of the invention also extends to a method of treating PRAME positive cancer in a patient comprising administering a TCR-anti-CD3 fusion molecule to said patient intravenously, wherein the TCR-anti-CD3 fusion molecule comprises:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
wherein the method comprises administration of:
(a) at least one first dose;
(b) at least one second dose; and then
(c) at least one third dose in the range of from 60-400 μg,
wherein the second dose is higher than the first dose and the third dose is higher than the second dose, and
wherein doses are administered every 6-8 days.

Methods of treating PRAME positive cancer include administering a therapeutically effective amount of a TCR anti-CD3 fusion molecule.

The TCR anti-CD3 fusion molecule can be formulated in pharmaceutical compositions. These compositions can comprise, in addition to the TCR anti-CD3 fusion molecule, one or more pharmaceutically acceptable excipients, carriers, buffers, stabilizers, or other materials well known to those skilled in the art. It may be provided in unit dosage form, will generally be provided in a sealed container and may be provided as part of a kit. Such a kit would normally (although not necessarily) include instructions for use and may include a plurality of said unit dosage forms. The pharmaceutical composition may be in any suitable form for intravenous administration. Such compositions may be prepared by any method known in the art of pharmacy, for example by mixing the active ingredient with the carrier(s) or excipient(s) under sterile conditions.

Administration of the TCR anti-CD3 fusion molecule is preferably in a “therapeutically effective amount,” this being an amount sufficient to show benefit to the patient. In relation to the first aspect of the invention, the therapeutically effective amount comprises at least one first dose in the range of 5-40 μg, at least one second dose in the range of 15-80 μg, and at least one third dose in the range of 60-400 μg. In relation to the second aspect of the invention, the therapeutically effective amount comprises at least one first dose and/or at least one second dose as determined by a clinician taking account of the state of disease and the condition of the patient being treated. In relation to the second aspect of the invention, the therapeutically effective amount comprises at least one third dose in the range of 60-400 μg.

Administration of the TCR anti-CD3 fusion molecule to the patient may result in an improved outcome for the patient. For example, an increased duration of progression free survival or overall survival. Administration of the TCR anti-CD3 fusion molecule to the patient may result in decrease in overall tumor size as determined by RECIST v1.1 criteria (Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009; 45 (2): 228-247).

During the course of a clinical trial, including following administration of the TCR anti-CD3 fusion molecule to the patient, a patient may have a partial response (PR), a complete response (CR), or be identified as having stable disease (SD), or progressive disease (PD).

Preferred features of each aspect of the invention are as for each of the other aspects mutatis mutandis. It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the TCR-anti-CD3 fusion molecule for use and method for treating PRAME positive cancer are specifically embraced by the present invention and are disclosed herein just as if each and every combination was individually and explicitly disclosed. The published documents mentioned herein are incorporated to the fullest extent permitted by law. Citation or identification of any document in this application is not an admission that such document is available as prior art to the present invention.

### DESCRIPTION OF THE DRAWINGS

FIG. 1 shows the dose escalation cohorts described in the Examples. The first panel shows dose escalation cohorts for 37 patients and the second panel shows dose escalation cohorts for 55 patients, as documented at a later time point. Each box represents a separate cohort. Doses are represented as “X/Y/Z mcg”, where X is the first dose (in micrograms), Y is the second dose (in micrograms) and Z is the third dose (in micrograms). The first dose was administered on day 1, the second dose on day 8 and the third dose on day 15.

FIG. 2 shows the number of patients enrolled in each cohort and their type of cancer. In the first panel, a total of 36 patients were enrolled. In the second panel a total of 55 patients were enrolled.

FIG. 3 shows a summary of the adverse events (AEs) associated with administration of IMC-F106C across dose escalation cohorts where 37 patients had been enrolled.

FIG. 4 shows pharmacodynamic activity of IMC-F106C with 37 patients enrolled with respect to absolute lymphocyte count (ALC) and body temperature.

FIG. 5 shows pharmacodynamic activity of IMC-F106C with 37 patients enrolled with respect to production of cytokines (A) IL-6 and IFNg.

FIG. 6 shows the percentage change in target lesion size over time after administration with IMC-F106C. This figure indicates which cohort the patient belonged to (according to cohort defined dose, CDD, i.e., their third dose)

FIG. 7 shows the percentage change in target lesion size for individual patients after administration with IMC-F106C. This figure indicates which cohort the patient belonged to (according to CDD) and their cancer type. Cutaneous Melanoma=CM; Metastatic Uveal Melanoma=mUM; Serous Ovarian Carcinoma=sOC; Endometrial Carcinoma=EC.

FIG. 8 shows the percentage change in individual target lesion sizes after administration with IMC-F106C relative to cohort (according to CDD).

FIG. 9 shows a summary of the adverse events (AEs) associated with administration of IMC-F106C with 55 patients enrolled. The AEs are listed in two separate categories, those associated with having received a third dose in the range of 0.3-10 mcg and those associated with having received a third dose in the range of 20-320 mcg. Events marked with a * include events reported as a sign or symptom of CRS. The † sign denotes safety presented by an intended escalation target dose on Day 15. One out of 37 patients received a single dose of 2 mcg and did not reach target dose of ≥20 mcg.

FIG. 10 shows pharmacodynamic activity of IMC-F106C with respect to IFNgamma induction in peripheral blood; and lymphocyte count measurements in peripheral blood.

FIG. 11 shows the best % change from baseline with respect to RECIST responses for different tumour types. The ovarian cancer patient with unconfirmed PR (uPR), marked with a † sign, remained on treatment at the time of analysis and remained eligible for confirmation. PRAME expression in the tumours, as indicated by a ‡ sign, was assessed by immunohistochemistry (IHC) H-score. In the figure “Endo” corresponds to endometrial carcinoma; “NSCLC” corresponds to non-small cell lung carcinoma; and “TNBC” corresponds to triple-negative breast cancer. Two patients (1 with NSCLC, 1 serous ovarian as denoted by the * sign) discontinued treatment due to progressive disease (PD).

FIG. 12 is a spider plot showing the percentage change in target lesion size over time (by week) relative to a baseline measurement for the different tumors indicated. Each line represents the change over time for an individual patient. NSCLC corresponds to non small cell lung carcinoma.

FIG. 13 shows the best log reduction in circulating tumor DNA in 20 evaluable patients from the IMC-F106C clinical trial. ctDNA was assessed, as described in a PCT application entitled “Compositions and Methods for Treating Cancer that Demonstrates Decreasing Levels of ctDNA in a Subject”, filed on 31 Aug. 2022, and hereby incorporated by reference, using one of two defined panels depending on tumor type: a custom panel comprising GNAQ, GNA11, SF3B1, PLCB4, CYSLTR2, and EIF1AX, for uveal melanoma, or the G360™ panel comprising 73 genes frequently mutated in diverse cancers (Guardant Health, Inc. The tumour types are identified as follows: B, triple-negative breast cancer; C, cutaneous melanoma; ctDNA, circulating tumor DNA; E, endometrial carcinoma; LA, non-small cell lung adenocarcinoma; LS, non-small cell lung squamous cell carcinoma; O, ovarian; U, uveal melanoma; CPI, checkpoint inhibitor; tebe, tebentafusp.

The invention is further described in the following non-limiting Examples.

### EXAMPLE 1: IMC-F106C-101 CLINICAL TRIAL DESIGN

This Example is a summary of interim results at different timepoints from a Phase 1/2 multicenter, open-label, first-in-human dose escalation study (“IMC-F106C-101”) of IMC-F106C in HLA-A*02:01-positive participants with advanced cancers that are positive for PRAME. This study was designed to assess the safety, tolerability, pharmacokinetics (PK), immunogenicity, pharmacodynamics, and antitumour activity of IMC-F106C as a monotherapy and in combination with a checkpoint inhibitor (eg, atezolizumab, pembrolizumab) or in combination with a chemotherapy (e.g., gemcitabine, nab-paclitaxel, or PLD).

IMC-F106C is an Immune-mobilizing monoclonal T-cell receptor Against Cancer (ImmTAC®), a bispecific protein therapeutic comprising a soluble, affinity-enhanced T-cell receptor (TCR; targeting domain) fused to an antibody single-chain fragment variable that specifically recognizes cluster of differentiation 3 (anti-CD3 scFv; effector domain). IMC-F106C is described in WO 2018/234319, which is incorporated herein by reference herein in its entirety. In WO 2018/234319, IMC-F106C is designated as ImmTAC2.

The IMC-F106C TCR recognizes a complex consisting of a peptide fragment of preferentially expressed antigen in melanoma (PRAME) presented by human leukocyte antigen-A allele 02:01 (HLA-A*02:01). Once the soluble TCR is engaged, the effector domain can bind to CD3 on any T cell, stimulating the T cell to release effector cytokines and lyse the bound target cell. In addition, IMC-F106C-mediated tumour cell killing may prime an endogenous antitumour immune response.

PRAME is a cancer-testis antigen that is frequently highly expressed in a range of solid and hematologic malignancies including melanoma, ovarian carcinoma, uterine carcinoma, small-cell and non-small cell lung cancer, triple-negative breast cancer, and urothelial carcinoma.

The data described below were obtained at different time points with successive cohorts in IMC-F106C-101, including at a first timepoint from 36 patients treated in the dose escalation phase of the monotherapy arm of IMC-F106C-101, across multiple dose cohorts, and at a different timepoint from 55 patients.

IMC F106C was administered via weekly (Q1W) IV infusion in 21-day cycles. Each cycle contained a first dose on day 1, a second dose on day 8 and a third dose on day 15. The duration of IV infusion was typically 1 hour+10 minutes in Cycles 1 and 2 and 30 minutes (+10 minutes) starting at Cycle 3 Day 1.

Safety assessments included physical examination, vital signs, weight, Eastern Cooperative Oncology Group (ECOG) performance status, hematology, chemistry, coagulation, urinalysis, thyroid function, cytokine testing, pregnancy testing, cardiac testing, as well as Adverse Events (AE) collection. Adverse Events (AEs) were graded according to National Cancer Institute Common Terminology Criteria for Adverse Events (NCI CTCAE) v5.0 unless otherwise specified

Tumor response was determined locally according to Response Evaluation Criteria in Solid Tumors (RECIST) v1.1.

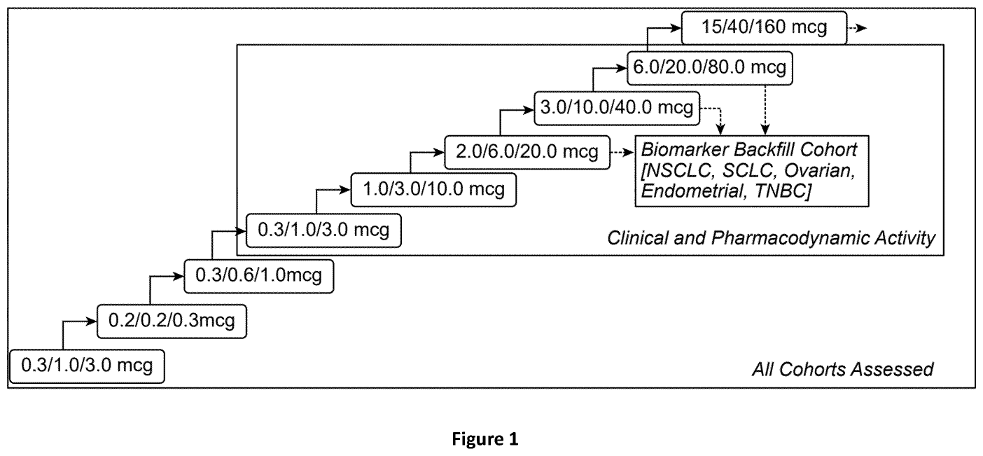

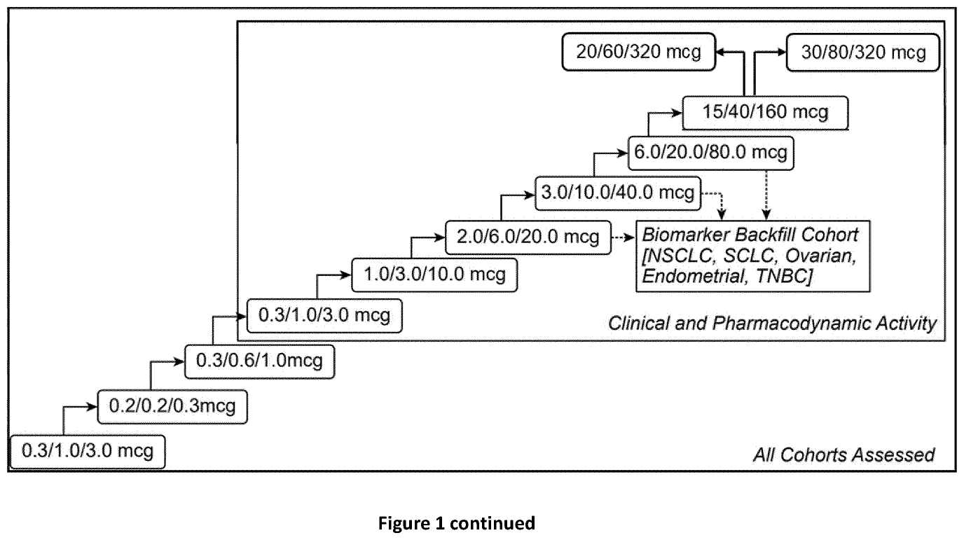

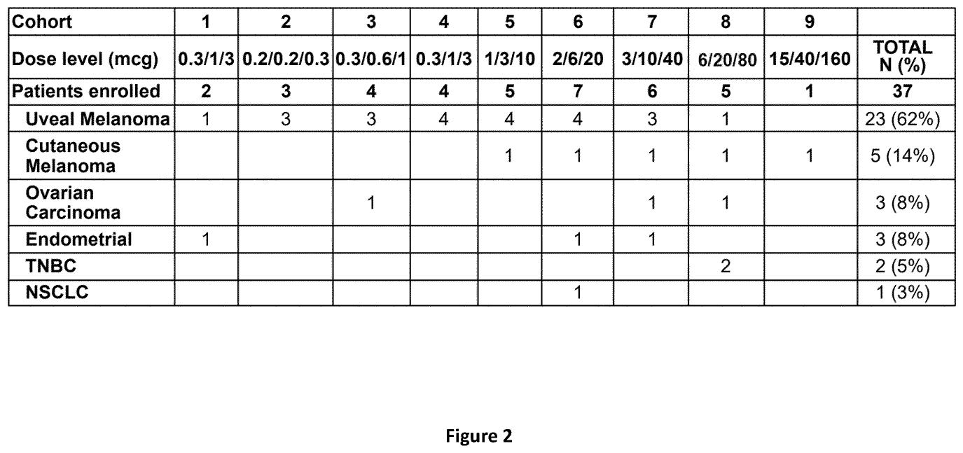

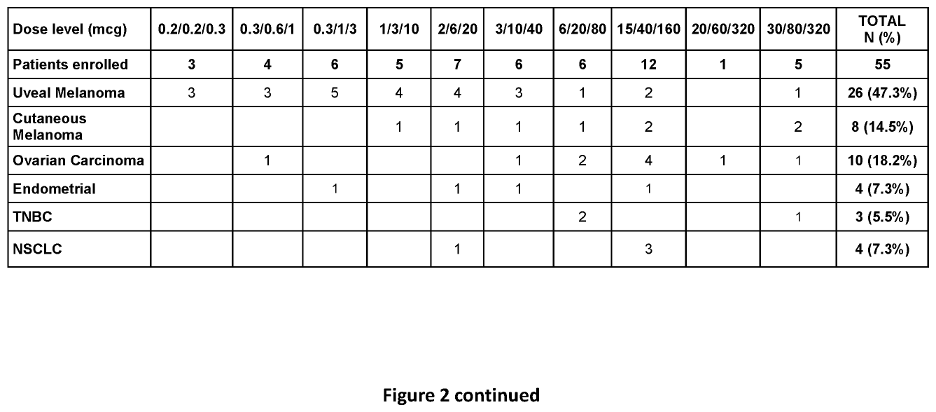

FIG. 1 shows the dose escalation cohorts. The first panel shows dose escalation cohorts for 37 patients and the second panel shows dose escalation cohorts for 55 patients, as assessed at a later time point. Each box represents a separate cohort. Doses are represented as “X/Y/Z mcg”, where X is the first dose (in micrograms), Y is the second dose (in micrograms) and Z is the third dose (in micrograms). The first dose was administered on day 1, the second dose on day 8 and the third dose on day 15. The second panel of FIG. 1 shows that further escalation to dosages of 30/80/320 mcg was conducted because the 15/40/160 mcg regimen indicated in the first panel of FIG. 1 was well tolerated and demonstrated pharmacodynamic and clinical activity. FIG. 2 shows the number of patients enrolled in each cohort and their type of cancer.

### EXAMPLE 2: IMC-F106C-101 SAFETY DATA AND PHARMACODYNAMIC ACTIVITY

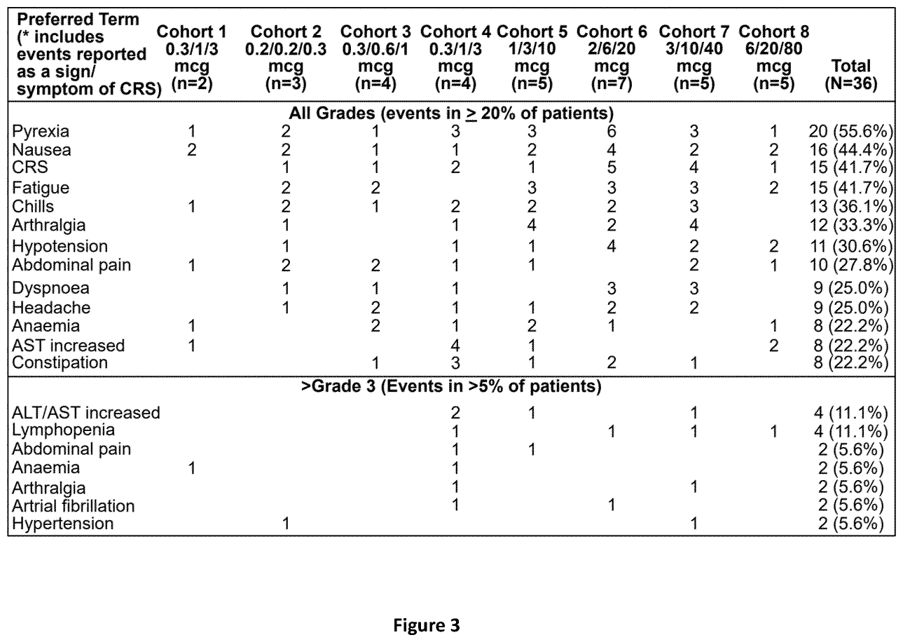

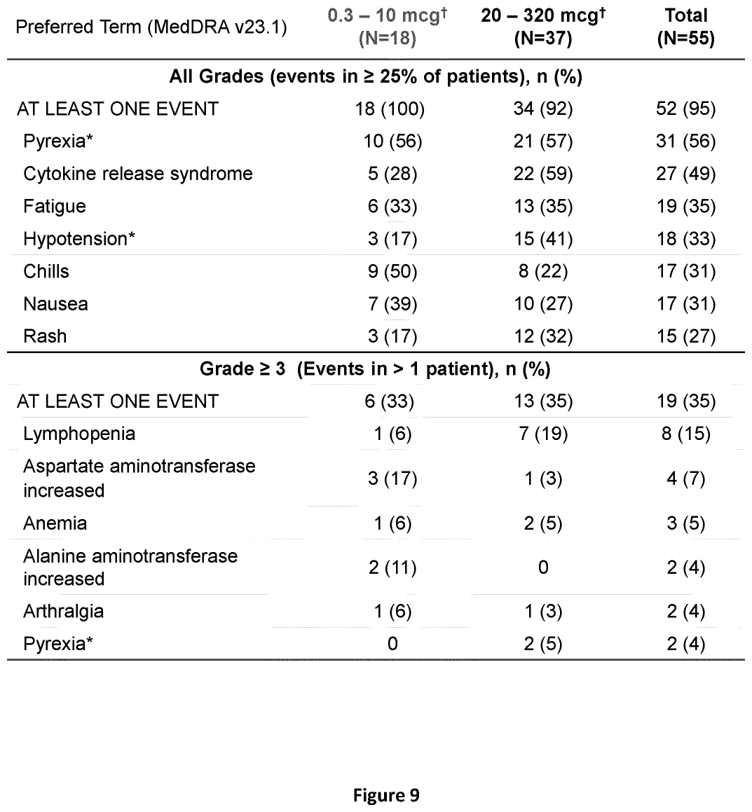

FIG. 3 shows a summary of the adverse events (AEs) associated with administration of IMC-F106C following dosing of 37 patients. FIG. 9 shows a summary of AEs following dosing of 55 patients. IMC-F106C was well tolerated in all cohorts. The most frequent AEs were cytokine mediated events such as pyrexia, cytokine release syndrome (CRS), chills, and nausea. There was no treatment discontinuation or death due to a treatment emergent AE or grade 5 events. One patient in Cohort 5 experienced a dose limiting toxicity (DLT), which was a grade 2 increase in aspartate transaminase at 1 mcg dose on C1D1. The analysis across 55 patients showed that the CRS events were manageable with the majority occurring within the first three doses. Of the CRS events across 55 patients, 71% were grade 1, 29% were grade 2 and there were no CRS events equal to or above grade 3. The AEs attenuated over time. In addition, no other immune related AEs typically seen following treatment with checkpoint inhibitors was observed. AEs also decreased in frequency with repeated dosing.

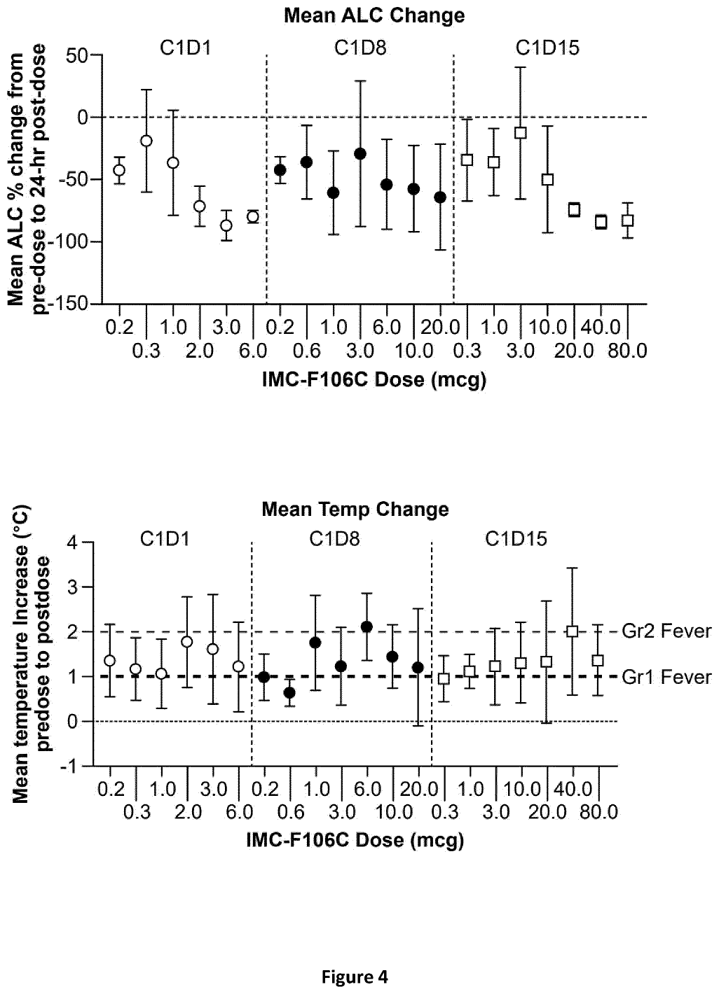

FIG. 4 shows pharmacodynamic activity of IMC-F106C with respect to absolute lymphocyte count (ALC) and body temperature. This figure shows that an ALC drop was observed progressively across all dose levels in the first 37 patients enrolled. Cohorts receiving a third dose of 40 mcg or above had a mean ALC drop of >80% during cycle 1. Also, body temperature elevation was observed across all dose levels. Patients in the Jun. 20, 1980 mcg cohort were given a steroid-based premedication.

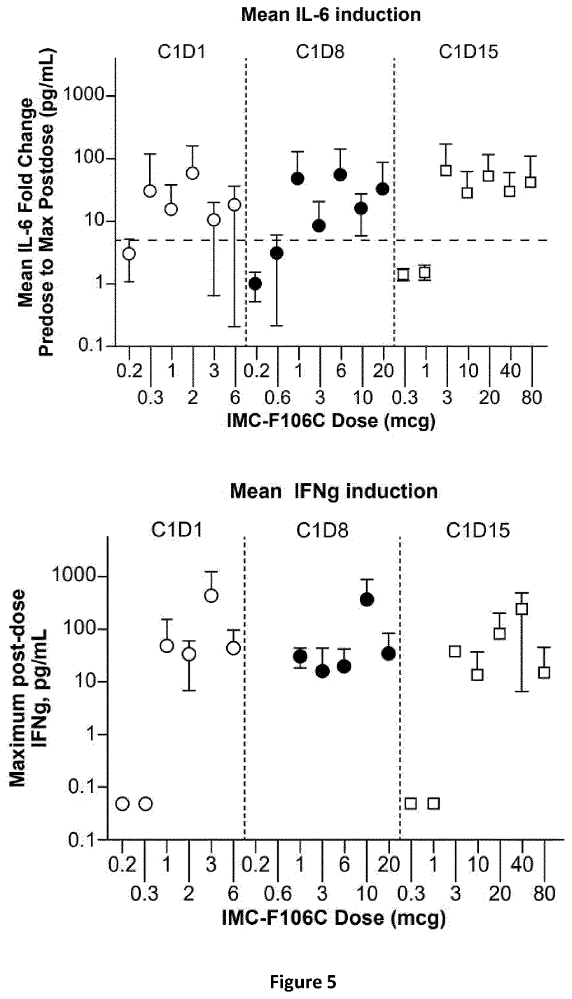

FIG. 5 shows pharmacodynamic activity of IMC-F106C with respect to production of cytokines IL-6 and IFNg. This Figure shows that all of the first 37 patients enrolled receiving a third dose of at least 3 mcg displayed a greater than five-fold increase in IL-6 production. Similarly, IFNg production was induced in patients receiving at least 3 mcg. Patients in the Jun. 20, 1980 mcg cohort were given a steroid-based premedication.

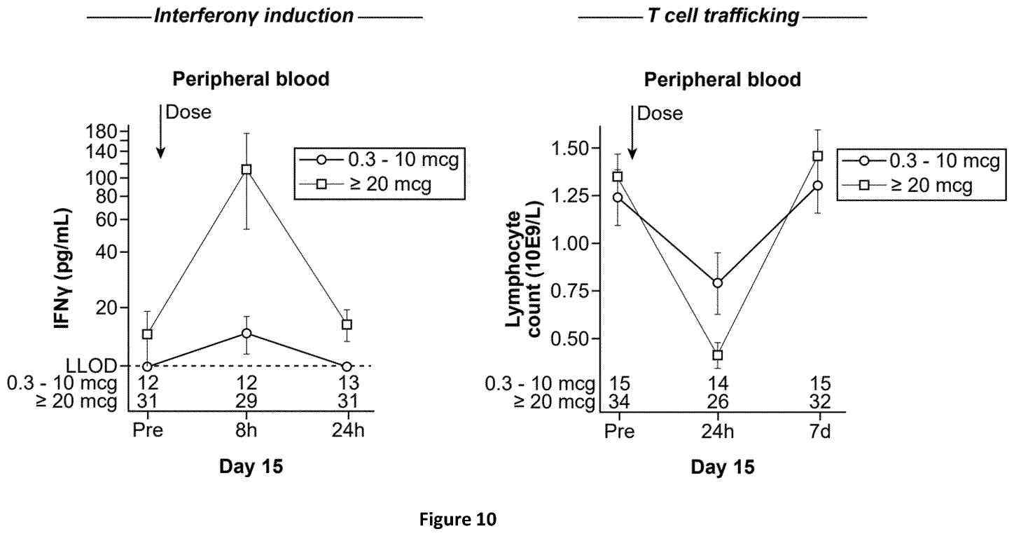

FIG. 10 demonstrates a strong and consistent pharmacodynamic activity at greater than or equal to 20 mcg IMC-F106C. In particular, administration of doses of 20 mcg and above resulted in consistent and robust IFN gamma induction, a specific marker of T cell activation. Administration of doses of 20 mcg and above also resulted in a corresponding reduction of lymphocyte count from the peripheral blood, and an increase in T cell infiltration as early as day 28 into the tumor site following treatment with IMC-F106C has been observed. These observations indicate that administration of IMC-F106C at these doses redirected T cells into the tumor.

### EXAMPLE 3: IMC-F106C-101 CLINICAL EFFICACY AND DURABILITY

Interim clinical efficacy data across cohorts 1 to 8 (the first 37 patients enrolled) (only one patient was evaluated in cohort 8 and this patient did not proceed past the third dose, C1D15) is shown in Table 2, below:

| Best Response | Total Evaluable (N = 31) ^{†} n (%) | |
|---|---|---|
| Partial Response (confirmed) | 2 | (7) |
| Stable Disease | 10 | (32) |
| Progressive Disease | 19 | (61) |
| Reason for progression | | |
| Target lesion | 15 | (79) |
| Non-target lesion | 4 | (21) |
| New lesion^{#} | 0 | (0)^{#} |

^{†} Scan data not available for 6 patients.

Table 2 shows that even at such low doses of IMC-F106C, two patients had a partial response to treatment. Patients that had a partial response or stable disease (RECIST) were observed in patients receiving a third dose of 20 mcg or higher. Patients that had progressive disease were mostly from cohorts receiving a third dose lower than 20 mcg.

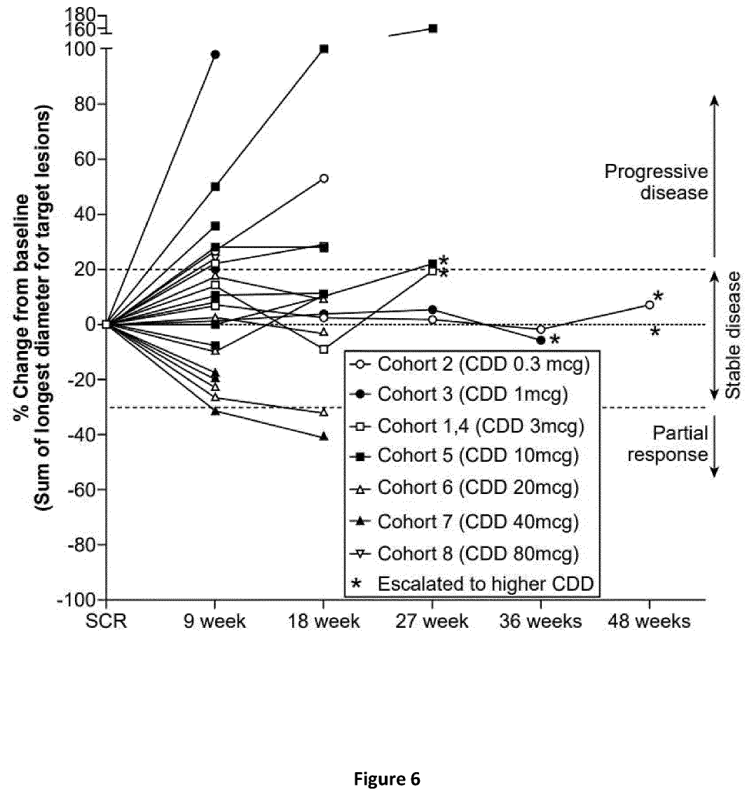

FIG. 6 shows the percentage change in target lesion size over time after administration with IMC-F106C in cohorts 1 to 8 (only one patient was evaluated in cohort 8 and this patient did not proceed past the third dose, C1D15). This figure shows that, even at such low dosages of IMC-F106C, disease stabilization was achieved for a long duration (i.e., 27 weeks and above) in some patients and that there is a correlation between tumour shrinkage and higher doses. Also, the majority of patients with stable disease displayed a reduction in longest diameter of the target lesions.

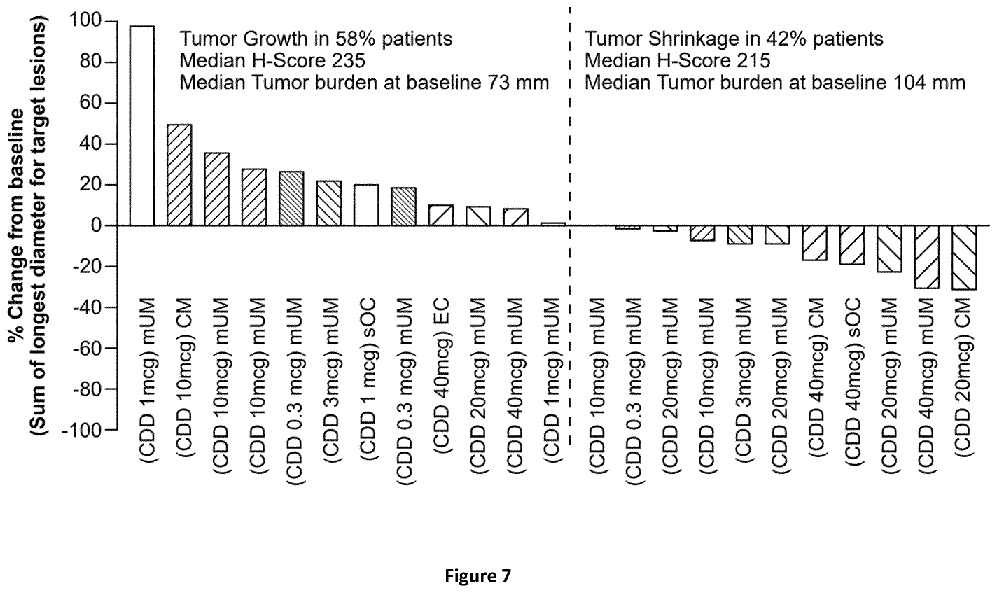

FIG. 7 shows the percentage change in target lesion size after administration with IMC-F106C. This figure indicates which cohort the patient belonged to and their cancer type. The dashed line separates patients that had tumour shrinkage vs those that did not. Tumour shrinkage was observed in 42% of patients and was correlated with higher doses of IMC-F106C. The majority of patients with tumour shrinkage were melanoma patients (largest enrolled tumour type). However, shrinkage in ⅓ ovarian cancer patients was observed as well.

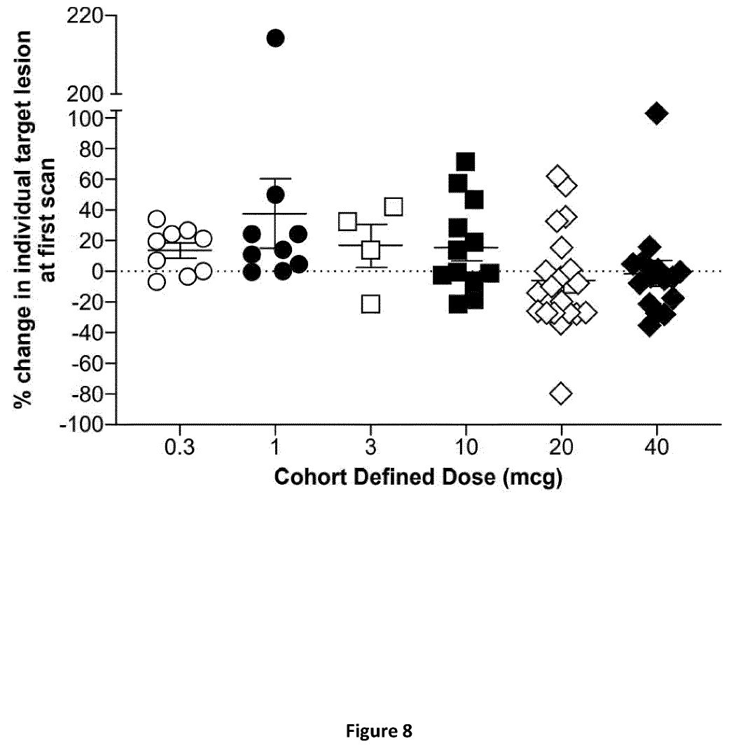

FIG. 8 shows the percentage change in individual target lesion sizes after administration with IMC-F106C relative to cohort.

FIGS. 7 and 8 show that tumour shrinkage was more common for patients receiving a third dose of 20 mcg or higher. This is despite high tumour burden at screening. Most of these patients experience shrinkage of the majority of their target lesions. Nearly all patients have a high PRAME H-score with median H-Scores >200 in both tumour shrinkage/tumour growth groups. In the efficacy population cohorts with 55 patients enrolled, the median H score was 188 out of 300. Interestingly, in patients who experienced tumour shrinkage, median tumour burden at baseline was 104 mm, indicating some patients with larger tumour lesions at baseline are also deriving clinical benefit from IMC-F106C. There was no correlation between tumour burden at base line (according to sum of longest diameter for target lesions) and change in tumour size after administration with IMC-F106C.

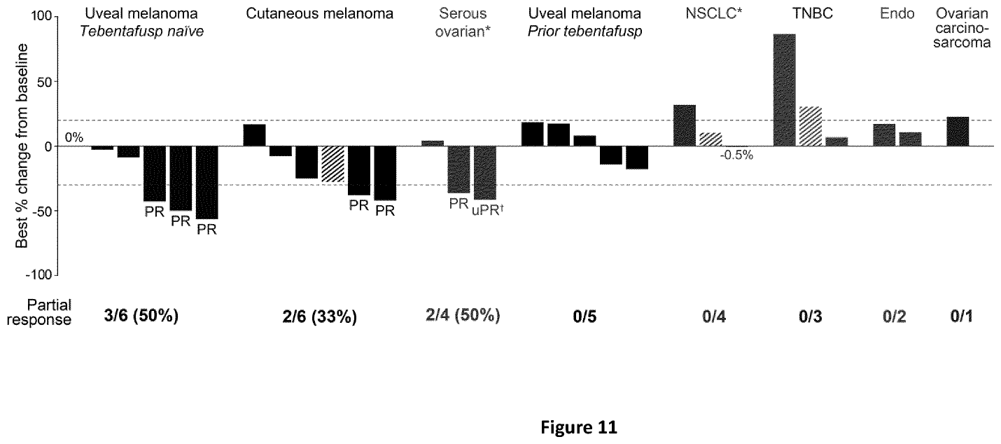

FIG. 11 shows the RECIST responses in patients across successive cohorts with 55 patients enrolled in multiple tumors. In uveal melanoma, partial responses (PRs) were observed in 50% of evaluable patients (3/6 PRs); in cutaneous melanoma patients all of whom had prior anti-PD1 and ipilumumab treatment, PRs were observed in 33% of evaluable patients (2/6); in platinum-resistant serous ovarian, PRs were observed in 50% of evaluable patients (2/4). It was notable to see responses even in patients who progressed on prior immunotherapy.

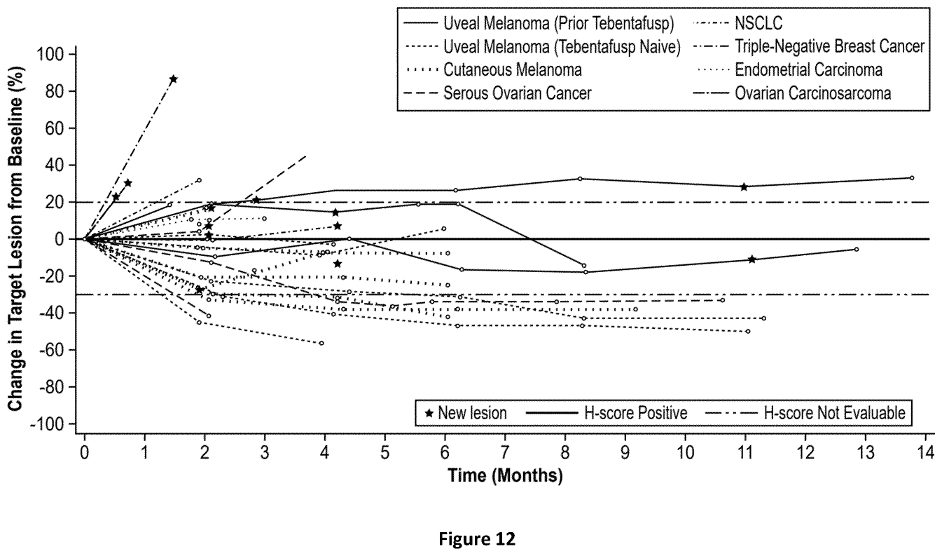

FIG. 12 demonstrates that IMC-F106C resulted in clinical activity in various tumor types. In particular, patients with partial response (PR) have promising durability as indicated by stabilization or reduction of lesion size over time. A number of patients without PR have long term disease stabilization.

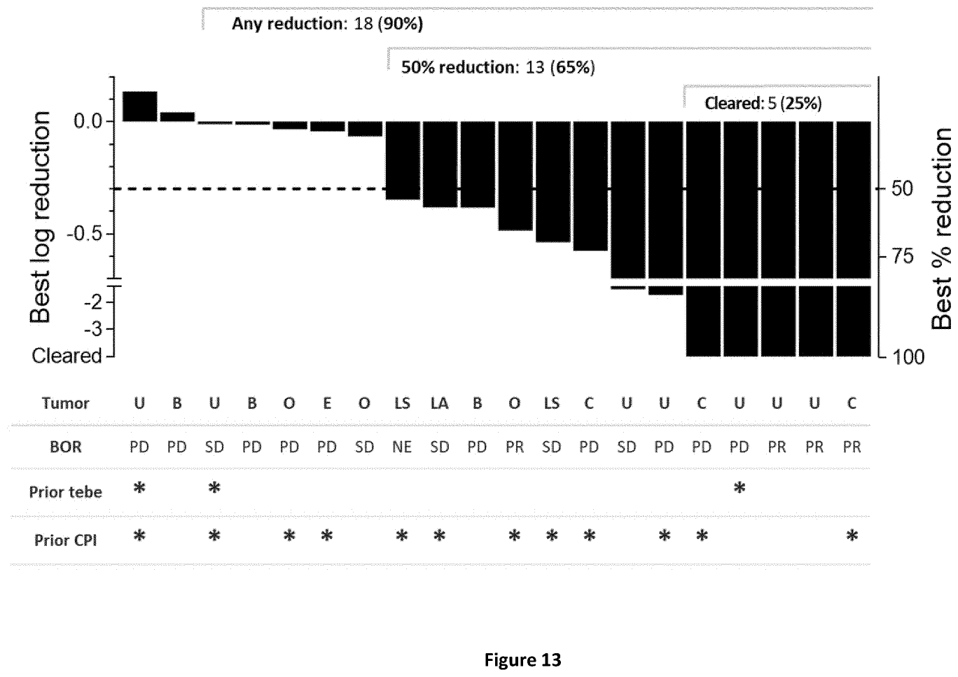

FIG. 13 shows that a reduction in circulating tumor DNA was observed across different tumor types. ctDNA reduction is an emerging early marker of clinical benefit in the IMC-F106C-101. Of 20 evaluable patients, nearly all had a reduction in ctDNA, a majority had 50% reduction and 25% cleared their ctDNA. Four PR patients evaluated for ctDNA had 50% or greater ctDNA reduction including 3 with complete clearance. The reduction and clearance of ctDNA was generally observed with one to two months of treatment.

### Summary of Results

The results described above are the first demonstration of the therapeutic potential of a soluble bispecific TCR targeting PRAME. IMC-F106C exhibits pharmacodynamic and clinical activity as shown by the results of the IMC-F106C-101 clinical trial.

The results described herein show that IMC-F106C was well-tolerated in humans. In particular, a first dose of 6 mcg on day 1, a second dose of 15 mcg on day 8 and a third dose of 80 mcg on day 15 (6/15/80 mcg cohort) was well tolerated, supporting further escalation to higher dosages of, for example 15/40/160 mcg and 20/60/240 mcg or higher. When IMC-F106C was administered at higher dosages of 15/40/160 mcg and 30/80/320 mcg it was also well tolerated and clinical activity was observed in both cohorts.

Administration of IMC-F106C (also referred to as PRAMExCD3 ImmTAC) activated T cells. IMC-F10C was well-tolerated, with CRS mostly Grade 1, and no Grade ≥3, and occurring predominantly during the initial three doses. The IMC-F106C-associated treatment-related adverse events were manageable; with no events having led to discontinuation or death. There was a consistent and strong pharmacodynamic biomarker activity observed at a third dose or cohort designated dose (CDD) of 20 mcg and above of IMC-F106C. In some cases, at a third dose or CDD of 20 mcg and above, clinical activity was also observed.

Treatment with IMC-F106C has led to durable (up to 9+ months) RECIST PRs across multiple tumor types including in cutaneous melanoma patients who had progressed following prior anti-PD1 and anti-CTLA4, in heavily pre-treated, platinum-resistant ovarian carcinoma patients; and in uveal melanoma patients. There has also been an observed benefit in disease control, including conversion of stable disease (SD) to PR. Almost all of the evaluable patients, across multiple tumor types, also presented with a reduction in ctDNA, where early reduction appeared to be associated with clinical benefit. Complete ctDNA clearance appeared common in melanoma patients receiving effective treatment.

1. A TCR-anti-CD3 fusion molecule comprising:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
for use in a method of treating PRAME positive cancer in a patient comprising administering the TCR-anti-CD3 fusion molecule to said patient intravenously, wherein the method comprises administration of:
(a) at least one first dose in the range of from 5-40 μg;
(b) at least one second dose in the range of from 15-80 μg; and then
(c) at least one third dose in the range of from 60-400 μg,
wherein the second dose is higher than the first dose and the third dose is higher than the second dose, and
wherein doses are administered every 6-8 days.

2. The TCR-anti CD3 fusion molecule for use according to claim 1, wherein the TCR-anti-CD3 fusion molecule comprises an alpha chain amino acid sequence corresponding to SEQ ID NO: 14 and a TCR beta chain-anti-CD3 amino acid sequence corresponding to SEQ ID NO: 16.

3.-4. (canceled)

5. The TCR-anti-CD3 fusion molecule for use according to claim 1, wherein the first dose is in the range of from 10-20 μg, the second dose is in the range of from 30-50 μg and the third dose is in the range of from 100-200 μg.

6.-8. (canceled)

9. The TCR-anti-CD3 fusion molecule for use according to claim 1, wherein the first dose is in the range of from 10-30 μg and the second dose is in the range of from 40-70 μg.

10. The TCR-anti-CD3 fusion molecule for use according to claim 1, wherein the first dose is in the range of from 10-30 μg, the second dose is in the range of from 40-70 μg and the third dose is at least 150 μg.

11. The TCR-anti-CD3 fusion molecule for use according to claim 1, wherein the first dose is in the range of from 10-30 μg, the second dose is in the range of from 40-70 μg and the third dose is in the range of from 150-400 μg.

12. The TCR-anti-CD3 fusion molecule for use according to claim 1, wherein the first dose is in the range of from 10-30 μg, the second dose is in the range of from 40-70 μg and the third dose is in the range of from 150-330 μg.

13. The TCR-anti-CD3 fusion molecule for use according to claim 1, wherein the first dose is in the range of from 10-30 μg, the second dose is in the range of from 40-70 μg and the third dose is in the range of from 150-170 μg.

14. The TCR-anti-CD3 fusion molecule for use according to claim 1, wherein the first dose is in the range of from 10-30 μg, the second dose is in the range of from 40-70 μg and the third dose is 160 μg.

15.-30. (canceled)

31. The TCR-anti-CD3 fusion molecule for use according to claim 1, wherein a further third dose is administered every 6-8 days until treatment is stopped.

32. The TCR-anti-CD3 fusion molecule for use according to claim 1, wherein a steroid is administered prior to the first, second and/or third dose.

33. The TCR-anti-CD3 fusion molecule for use according to claim 1, which is administered in combination with one or more anti-cancer therapies.

34. The TCR-anti-CD3 fusion molecule for use according to claim 33, wherein the anti-cancer therapy is a checkpoint inhibitor.

35. The TCR-anti-CD3 fusion molecule for use according to claim 34, wherein the checkpoint inhibitor is selected from the group consisting of: atezolizumab, pembrolizumab, nivolumab, avelumab, durvalumab, ipilimumab and tremelimumab.

36. The TCR-anti-CD3 fusion molecule for use according claim 1, which is administered in combination with another TCR-anti-CD3 fusion molecule, wherein the other TCR-anti-CD3 fusion molecule comprises a TCR that binds to a gp100 peptide-MHC complex.

37. The TCR-anti-CD3 fusion molecule for use according to claim 36, wherein the other TCR-anti-CD3 fusion molecule is tebentafusp.

38. The TCR-anti-CD3 fusion molecule for use according to claim 1, wherein the PRAME positive cancer is selected from the group consisting of melanoma, lung cancer, breast cancer, ovarian cancer, endometrial cancer, esophageal cancer, bladder cancer, head and neck cancer, uterine cancer, Acute myeloid leukemia, chronic myeloid leukemia, and Hodgkin's lymphoma.

39. The TCR-anti-CD3 fusion molecule for use according to claim 38, wherein the melanoma is uveal melanoma or cutaneous melanoma, the lung cancer is non-small cell lung cancer (NSCLC) or small cell lung cancer (SCLC), or the breast cancer is triple-negative breast cancer (TNBC).

40. A method of treating PRAME positive cancer in a patient comprising administering a TCR-anti-CD3 fusion molecule to said patient intravenously, wherein the TCR-anti-CD3 fusion molecule comprises:
a TCR alpha chain amino acid sequence of SEQ ID NO: 14 or a TCR alpha chain amino acid sequence that has at least 90%, at least 95% or 100% identity to the amino acid sequence of SEQ ID NO: 14, and
a TCR beta chain-anti-CD3 amino acid sequence of SEQ ID NO: 16 or a TCR beta chain-anti-CD3 amino acid sequence that has at least 90%, at least 95% or 100% identity to the amino acid sequence of SEQ ID NO: 16,
wherein the TCR alpha chain variable domain comprises CDRs 1, 2 and 3 having the amino acid sequences of SEQ ID NOs: 3, 4 and 5 respectively and the TCR beta chain variable domain comprises CDRs 1, 2 and 3 having the amino acid sequences of SEQ ID NOs: 9, 10 and 11 respectively,
wherein the method comprises administration of:
(a) at least one first dose in the range of from 5-40 μg;
(b) at least one second dose in the range of from 15-80 μg; and then
(c) at least one third dose in the range of from 60-400 μg,
wherein the second dose is higher than the first dose and the third dose is higher than the second dose, and
wherein doses are administered every 6-8 days.


SEQUENCE LISTING (30 sequences)

SEQ ID NO: 1 - protein, 9 aa, Homo sapiens
SLLQHLIGL

SEQ ID NO: 2 - protein, 115 aa, synthetic construct
GDAKTTQPNSMESNEEEPVHLPCNHSTISGTDYIHWYRQLPSQGPEYVIHGLTSNVNNRMASLAIAEDRKSSTLILHRATLRDAAVYYCILILGHSRLGNYIATFGKGTKLSVIP

SEQ ID NO: 3 - protein, 7 aa, synthetic construct
TISGTDY

SEQ ID NO: 4 - protein, 5 aa, synthetic construct
GLTSN

SEQ ID NO: 5 - protein, 17 aa, synthetic construct
CILILGHSRLGNYIATF

SEQ ID NO: 6 - protein, 17 aa, synthetic construct
IHWYRQLPSQGPEYVIH

SEQ ID NO: 7 - protein, 33 aa, synthetic construct
VNNRMASLAIAEDRKSSTLILHRATLRDAAVYY

SEQ ID NO: 8 - protein, 114 aa, synthetic construct
DGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVT

SEQ ID NO: 9 - protein, 5 aa, synthetic construct
LNHDA

SEQ ID NO: 10 - protein, 7 aa, synthetic construct
SQIMGDE

SEQ ID NO: 11 - protein, 15 aa, synthetic construct
CASSWWTGGASPIRF

SEQ ID NO: 12 - protein, 17 aa, synthetic construct
MYWYRQDPGQGLRLIYY

SEQ ID NO: 13 - protein, 35 aa, synthetic construct
QKGDIAEGYSVSREKKESFPLTVTSAQKNPTAFYL

SEQ ID NO: 14 - protein, 201 aa, synthetic construct
GDAKTTQPNSMESNEEEPVHLPCNHSTISGTDYIHWYRQLPSQGPEYVIHGLTSNVNNRMASLAIAEDRKSSTLILHRATLRDAAVYYCILILGHSRLGNYIATFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 15 - protein, 86 aa, synthetic construct
NIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 16 - protein, 502 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 17 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 18 - protein, 5 aa, synthetic construct
GGGGS

SEQ ID NO: 19 - protein, 130 aa, synthetic construct
EDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 20 - protein, 5 aa, synthetic construct
GGGSG

SEQ ID NO: 21 - protein, 5 aa, synthetic construct
GGSGG

SEQ ID NO: 22 - protein, 5 aa, synthetic construct
GSGGG

SEQ ID NO: 23 - protein, 6 aa, synthetic construct
GSGGGP

SEQ ID NO: 24 - protein, 5 aa, synthetic construct
GGEPS

SEQ ID NO: 25 - protein, 7 aa, synthetic construct
GGEGGGP

SEQ ID NO: 26 - protein, 12 aa, synthetic construct
GGEGGGSEGGGS

SEQ ID NO: 27 - protein, 26 aa, synthetic construct
GDAKTTQPNSMESNEEEPVHLPCNHS

SEQ ID NO: 28 - protein, 10 aa, synthetic construct
GKGTKLSVIP

SEQ ID NO: 29 - protein, 26 aa, synthetic construct
DGGITQSPKYLFRKEGQNVTLSCEQN

SEQ ID NO: 30 - protein, 9 aa, synthetic construct
GPGTRLTVT